(19) United States
(12) Patent Application Publication
(10) Pub. No.: US 2026/0285783 A1
(43) Pub. Date: Sep. 24, 2026

(54) CATALYSTS FOR LIGHT OLEFIN OLIGOMERIZATION

(71) Applicant: EXXONMOBIL TECHNOLOGY AND ENGINEERING COMPANY, Spring, TX (US)

(72) Inventors: Emiel DE SMIT, Sint-Lambrechts-Woluwe (BE); Michael P. LANCI, Flemington, NJ (US); Trong D. PHAM, Whitehouse Station, NJ (US)

(21) Appl. No.: 19/664,125

(22) Filed: Apr. 30, 2026

Related U.S. Application Data
(63) Continuation-in-part of application No. PCT/US2026/019792, filed on Mar. 18, 2026.
(63) Continuation-in-part of application No. PCT/US2026/019794, filed on Mar. 18, 2026.
(60) Provisional application No. 63/774,405, filed on Mar. 19, 2025.
(60) Provisional application No. 63/774,405, filed on Mar. 19, 2025.
(60) Provisional application No. 63/921,688, filed on Nov. 20, 2025.
(60) Provisional application No. 63/921,711, filed on Nov. 20, 2025.
(60) Provisional application No. 63/921,688, filed on Nov. 20, 2025.
(60) Provisional application No. 63/921,711, filed on Nov. 20, 2025.

(51) Int. Cl. C07C 2/12

(52) U.S. Cl. CPC C07C 2/12; C07C 2529/06

(57) ABSTRACT

Methods are provided for low temperature conversion of olefinic feeds that contain C₃-C₈ olefins, and further optionally contain C₂ olefins. It has been discovered that conversion of C₂ olefins and/or C₃-C₈ olefins can be performed by using catalysts containing zeolitic framework structures that have one or more beneficial features. This conversion can be achieved at relatively low reaction temperatures. In some aspects, the conversion can be achieved without the presence of a substantial amount of catalytic metal supported on the catalyst.

### FIELD

Methods are provided for low temperature conversion of feeds containing C₂ olefins and/or C₃-C₈ olefins to form distillate boiling range products.

### BACKGROUND

One potential pathway for converting biomass to fuels is by forming methanol and/or ethanol from biomass and converting the methanol and/or ethanol to olefins. The olefins can then be oligomerized to form fuels boiling range compounds, such as distillate (e.g. jet fuel/diesel) boiling range compounds. Unfortunately, a variety of challenges remain for performing such oligomerization processes.

One of the difficulties in converting olefins into distillate boiling range products is the need to use elevated temperatures to perform the conversion. Generally, increasing the temperature of the conversion process is beneficial for increasing the single pass conversion of the olefins. However, increasing the temperature also increases the costs of operating the reaction system. Additionally, higher operating temperatures tend to increase the number of side reactions and/or secondary reactions that can occur in parallel to the desired conversion reaction. Therefore, it would be desirable to have improved conversion methods that can operate at lower conversion temperatures.

U.S. Pat. No. 11,078,433 describes methods and materials for oligomerization of lower olefins to transportation fuels.

U.S. Patent Application Publication 20110005190 describes a kerosene base fuel including less than 15 wt % aromatics and 80 wt % or more aliphatic hydrocarbons. For the aliphatic hydrocarbons, 20 vol % or more are n-paraffins, and 25 vol % or more are cycloparaffins.

International Publication Number 2022/060353 describes MWW type zeolite catalysts. The catalysts are described in conjunction with processes for alkylation of isoparaffins with light olefins. The alkylation processes are described as being performed at isoparaffin to olefin volume ratios of 100 to 1 or greater.

U.S. Pat. Nos. 9,932,531 and 10,005,974 describe one-step and two-step processes for converting ethylene to fuels. The one-step oligomerization processes are described as requiring a temperature of 280° C. or greater. The two-step processes require a series of different catalysts for the oligomerization.

U.S. Pat. No. 4,433,185 describes performing olefin oligomerization over a limited group of zeolites including ZSM-5, ZSM-11, ZSM-12, ZSM-23, ZSM-35, and ZSM-38. It is noted that conversion of C₂ olefins is low when performing single stage oligomerization.

U.S. Pat. No. 7,183,450 describes performing olefin oligomerization over a variety of zeolitic framework structures. The framework structures include MFI, MEL, MTW, EUO, MTT, HEU, FER, AFO, AEL, and TON.

U.S. Pat. Nos. 4,542,251, 4,628,138 and 4,740,645 describe olefin oligomerization in the presence of catalyst corresponding to a catalytic metal or catalytic metal oxide supported on a zeolite substrate.

U.S. Pat. No. 4,227,992 describes oligomerization of olefin feeds in the presence of a ZSM-5 catalyst (MFI framework structure).

U.S. Pat. No. 5,600,048 describes alkylation and transalkylation of aromatic compounds with ethylene to form ethylbenzene.

### SUMMARY

In some aspects, a method of converting a feed containing C₂-C₈ olefins is provided. The method includes exposing a feed containing 50 wt % or more of C₂-C₈ olefins to a catalyst including a zeolitic framework structure having a 1-dimensional or 3-dimensional largest pore channel under conversion conditions to form a conversion product containing 70 wt % or more of 121° C.+ components and 2.0 wt % or less of C₃-C₆ alkanes, relative to a weight of olefins in the feed. The conversion conditions can include single pass conversion of 30 wt % or more of C₂ olefins and 80 wt % or more of C₄ olefins. A maximum diameter of a sphere that can be included in the largest pore channel is 5.0 Angstroms to 6.5 Angstroms. Optionally, the zeolitic framework structure can further include an elliptical 8-membered ring pore channel having a pore channel size of 2.2 Angstroms to 3.5 Angstroms for a minor elliptical axis and 4.0 Angstroms to 6.0 Angstroms for a major elliptical axis.

In other aspects, a method of converting a feed containing C₂-C₈ olefins is provided. The method includes exposing a feed comprising 5.0 wt % or more of C₂ olefins and 25 wt % or more of C₂-C₈ olefins to a catalyst including a zeolitic framework structure under conversion conditions to form a conversion product comprising 70 wt % or more of 121° C.+ components, relative to a weight of olefins in the feed. The conversion conditions can include single pass conversion of 50 wt % or more of C₂ olefins and 80 wt % or more of C₄ olefins. The catalyst further includes i) a zeolitic framework structure having an elliptical 8-membered ring pore channel having a pore channel size of 2.2 Angstroms to 3.5 Angstroms for a minor elliptical axis and 4.0 Angstroms to 6.0 Angstroms for a major elliptical axis, the zeolitic framework structure being a 2-dimensional or 3-dimensional zeolitic framework structure; ii) a zeolitic framework structure where a maximum diameter of a sphere that can be included in a largest pore channel is 4.0 Angstroms to 6.0 Angstroms; or iii) a combination of i) and ii).

In some aspects, the methods can further include exposing an oxygenate feed comprising one or more alcohols to a conversion catalyst under conversion conditions to form a conversion effluent, the feed containing at least a portion of the conversion effluent. In some aspects, the conversion effluent can include 5.0 wt % or more (or 10 wt % or more) of C₂ olefins and 50 wt % or more of C₂-C₈ olefins. In some aspects, the conversion effluent can include less than 10 wt % of C₂ olefins (or less than 5.0 wt %) and 50 wt % or more of C₂-C₈ olefins.

### BRIEF DESCRIPTION OF THE DRAWINGS

FIG. 1 shows example configurations for oligomerization of olefins to form fuel boiling range products.

FIG. 2 shows conversion of C₂ olefins versus the natural log of “1—the fractional weight concentration of C₄ olefins conversion” for various catalysts using a model feed for oligomerization.

FIG. 3A and FIG. 3B show conversion of C₂ olefins versus conversion of C₄ olefins for various catalysts using a model feed for oligomerization.

FIG. 4 shows C₃-C₆ alkane selectivity versus C₂ olefin conversion for various catalysts using a model feed for oligomerization.

FIG. 5 shows C₃-C₆ alkane selectivity versus the corrected temperature for 95 wt % conversion of C₂-C₆ olefins for various catalysts using a model feed for oligomerization.

FIG. 6A and FIG. 6B shows the corrected temperature for 95 wt % conversion of C₂-C₆ olefins versus catalyst life for various catalysts using a model feed for oligomerization.

FIG. 7 shows C₂ olefin conversion, C₃-C₆ saturates make, corrected temperature, and distillate selectivity for various catalysts using a model feed for oligomerization.

FIG. 8 shows C₂ olefin conversion versus distillate selectivity for various catalysts using a model feed for oligomerization.

FIG. 9 shows the C₃-C₆ saturates (alkanes) make, C₂ conversion, olefin conversion, naphtha selectivity, and distillate selectivity for oligomerization of the olefinic feed over various catalysts. Data averaged between 90 and 95% olefin conversion.

FIG. 10 shows the C₃-C₆ saturates (alkanes) make, C₂ conversion, olefin conversion, naphtha selectivity, jet selectivity, and heavy diesel selectivity for oligomerization of the olefinic feed over the catalysts shown in FIG. 9.

FIG. 11 shows C₃-C₆ alkane selectivity versus C₄ olefin conversion for various catalysts using a model feed containing C₃-C₈ olefins for oligomerization.

FIG. 12 shows distillate selectivity versus C₃-C₆ alkane selectivity for various catalysts using a model feed containing C₃-C₈ olefins for oligomerization.

FIG. 13 shows C₃-C₆ alkane selectivity versus reactor temperature for various catalysts using a model feed containing C₃-C₈ olefins for oligomerization.

FIG. 14 shows C₃-C₆ alkane selectivity versus the corrected temperature for 95 wt % conversion of C₄ olefins for another group of catalysts using a second model feed containing C₃-C₈ olefins for oligomerization

FIG. 15 shows the corrected temperature for 95 wt % conversion of C₃-C₆ olefins versus catalyst life for various catalysts using a model feed for oligomerization.

FIG. 16 shows the C₃-C₆ saturates (alkanes) make, C₄ conversion, naphtha selectivity, and distillate selectivity for oligomerization of the C₃-C₆ olefinic feed over various catalysts. Data averaged between 90 and 95% olefin conversion.

### DETAILED DESCRIPTION

All numerical values within the detailed description and the claims herein are modified by “about” or “approximately” the indicated value, and take into account experimental error and variations that would be expected by a person having ordinary skill in the art.

### Overview

In various aspects, methods are provided for low temperature conversion of olefinic feeds that contain C₃-C₈ olefins, and further optionally contain C₂ olefins, while also reducing or minimizing the formation of C₃-C₈ alkanes. It has been discovered that conversion of C₂ olefins and/or C₃-C₈ olefins can be performed while reducing or minimizing formation of light alkanes by using a catalyst having an MTW, MEL/MFI, MFS, MTT, and/or MFI framework structure as the conversion catalyst. Reducing light alkane formation is beneficial, as light alkanes represent a low value side product. Avoiding light alkane formation means that additional light olefins are available for potential recycle, thus allowing for an increase in the net yield of distillate range products from the oligomerization reaction. In various aspects, the conversion reaction can allow for conversion of 30 wt % or more, or 40 wt % or more, of C₂ olefins (single pass basis) and/or conversion of 80 wt % or more, preferably 90 wt % or more, of C₃-C₈ olefins and/or conversion of 80 wt % or more, preferably 90 wt % or more, or C₄ olefins. This conversion reaction can result in formation of a conversion effluent containing 60 wt % or more of 121° C.+ components, relative to a weight of olefins in the feed. Additionally, this conversion can be achieved at relatively low reaction temperatures. In some aspects, the conversion can be achieved without the presence of a substantial amount of catalytic metal supported on the catalyst.

In various additional aspects, methods are provided for low temperature conversion of olefinic feeds that contain a mixture of C₂ olefins and C₃-C₈ olefins. It has been discovered that conversion can be performed at high conversion rates for both C₂ olefins and C₃-C₈ olefins by using a catalyst having an MOR, MTT, TON, FER, RFE, and/or MFI framework structure as the conversion catalyst. This ability to effectively convert both C₂ olefins and C₃-C₈ olefins at high conversion rates can allow for improved production of distillate fuels from olefinic feeds. In various aspects, the conversion reaction can allow for conversion of 50 wt % or more of C₂ olefins (single pass basis) while also converting 80 wt % or more, preferably 90 wt % or more, of C₃-C₈ olefins, while also forming a conversion effluent containing 50 wt % or more of 121° C.+ components, relative to a weight of olefins in the feed. Additionally, this high rate of conversion with elevated yield of distillate products can be achieved at relatively low reaction temperatures. In some aspects, this high conversion rate can be achieved without the presence of a substantial amount of catalytic metal supported on the catalyst.

More generally, in various aspects, methods are provided for low temperature conversion of olefinic feeds that contain a mixture of C₂ olefins and C₃-C₈ olefins. It has been discovered that catalysts having one or more of the following features can be beneficial for conversion of C₂ and/or C₃-C₈ olefins: A) A 2-dimensional (2-D) or 3-dimensional (3-D) zeolitic framework structure that has a small elliptical 8-member ring channel connected to a 10-member ring or other moderately larger channel; B) A 1-dimensional (1-D) pore channel in the framework structure that provides effective confinement for C₂ olefins, but with pore channels that are too narrow for effective fast growth of C₄ olefin monomers; C) A framework structure containing either 1-D or 3-D pore channels with a maximum diameter of a sphere that can be included in a pore channel of the framework structure of 5.0 to 6.5 Angstroms; (D) Having a suitable particle size for the solid acid catalyst and/or a suitable diffusion length within the framework structure; and/or (E) A relatively low Si to Al molar ratio in the framework structure, such as a Si to Al molar ratio of 5 to 40, or 8 to 25.

It is noted that addition of a catalytic metal would not be inherently detrimental to the conversion process when using a solid acid catalyst. However, to avoid poisoning a metal-based catalyst, impurities such as CO would need to be removed from the ethylene-containing feed to sufficiently low levels. In aspects where conversion is performed using a catalyst that is substantially without a supported catalytic metal, the need to improve feed purity can be reduced, minimized, or eliminated, thus providing a substantial cost and energy savings for the overall process.

During hydrocarbon processing, a variety of conversion reactions are used where a hydrocarbon feedstock is exposed to a conversion catalyst in order to obtain a conversion product. When designing such a process for commercial use, the conditions for the conversion reaction are conventionally specified by providing two types of information. First, some type of conversion percentage for the feed is specified, such as the weight percentage of olefins that are converted per pass, or the weight percentage of C₄ olefins that are converted per pass. Because feeds with mixtures of feed components can vary, specifying a conversion percentage allows the conditions to be relevant for the specific feed that is available for conversion. Second, some type of operating envelope is specified for the start of run temperature, pressure, and space velocity to use while achieving the target conversion percentage. This combination of factors allows a commercial scale process to be started up that can achieve a target level of feed conversion while also having the starting operating envelope for temperature, pressure, and space velocity conditions that can achieve the conversion percentage.

Unfortunately, conversion of olefins during an oligomerization reaction also tends to result in formation of light (C₃-C₈)alkanes. Due to the lower reactivity of alkanes versus alkenes, any alkanes that form under the oligomerization conditions do not undergo further reaction. Therefore, any alkanes that are formed do not become larger via oligomerization. C₃-C₈ alkanes have too low of a boiling point to be incorporated into a distillate fuel. Thus, any C₃-C₈ alkanes represent molecules that are not in the desired boiling range while also not having sufficient reactivity to be upgraded to the desired boiling range. Because of this, reducing or minimizing formation of C₃-C₈ alkanes is beneficial, as avoiding formation of alkanes increases the amount of olefins that are converted during a single pass and/or allows olefins to be recycled for oligomerization in a subsequent pass.

It has been discovered that MTW, MEL/MFI, MFS, MTT, and/or MFI framework catalysts can convert both C₂ alkenes and/or C₃-C₈ alkenes at unexpectedly high levels of conversion while also reducing or minimizing formation of C₃-C₈ alkanes. More generally, it has been discovered that catalysts containing zeolitic framework structures with certain beneficial features can convert both C₂ alkenes and/or C₃-C₈ alkenes at unexpectedly high levels of conversion while also reducing or minimizing formation of C₃-C₈ alkanes.

It has further been discovered that being able to start at a lower temperature can generally reduce or minimize secondary or side reactions that generate lower value conversion products. For example, for conversion of an olefinic feed, lower processing temperatures can tend to reduce or minimize production of C₃-C₆ alkanes. Thus, a catalyst which can provide an unexpectedly low temperature of operation while achieving a target conversion level is beneficial.

An unexpectedly low starting temperature can also be beneficial in other ways. For example, in situations where a catalyst is used in a fixed bed environment, having an unexpectedly low start of run temperature increases the amount of temperature increase that is available to compensate for catalyst deactivation during the course of a run. Still other benefits of a low conversion temperature can be reduced operating costs, including reduced costs for fresh feed pre-heating as well as reduced costs for pre-heating of any recycle streams in the process.

It has been discovered that MOR, MTT, TON, FER, RFE and/or MFI framework catalysts can convert both C₂ alkenes and C₃-C₈ alkenes at unexpectedly high levels of conversion. Although many catalysts can provide high conversion of C₃-C₈ olefins, the ability to effectively convert both C₂ olefins does not necessarily correlate with the ability to convert C₃-C₈ olefins. The ability of MOR, MTT, TON, FER, RFE and/or MFI framework catalysts to provide high levels of conversion for both C₂ olefins and C₃-C₈ olefins can enable single stage oligomerization of olefinic feeds that contain C₂ olefins. More generally, it has been discovered that catalysts containing zeolitic framework structures with certain beneficial features can convert both C₂ alkenes and/or C₃-C₈ alkenes at unexpectedly high levels of conversion.

In various aspects, the conversion temperature can be 120° C. to 300° C., or 120° C. to 250° C., or 120° C. to 220° C., or 120° C. to 195° C., or 150° C. to 300° C., or 150° C. to 250° C., or 150° C. to 220° C., or 150° C. to 195° C. In such aspects, a mixed feed of C₂ olefins and C₃-C₈ olefins can be exposed to a catalyst having a suitable framework structure to achieve a C₂ olefin conversion rate of 30 wt % or more (or 40 wt % or more, or 50 wt % or more, or 60 wt % or more, or 70 wt % or more, or 80 wt % or more, such as up to 90 wt % or possibly still higher) while also achieving a C₃-C₈ olefin conversion rate of 80 wt % or more, or 90 wt % or more, or 95 wt % or more, such as up to substantially complete conversion (100 wt %). Additionally, in some aspects, the conversion of C₄ olefins can be 80 wt % or more, or 90 wt % or more, such as up to substantially complete conversion of C₄ olefins (100 wt %). In such aspects, the conversion reaction can have a selectivity for formation of distillate boiling range products of 40 wt % or more, or 50 wt % or more, or 60 wt % or more, or 70 wt % or more, such as up to 85 wt % or possibly still higher. Additionally or alternately, the selectivity for formation of diesel products (that boil above the distillate boiling range) can be reduced or minimized, so that the selectivity for diesel products is 25 wt % or less, or 20 wt % or less. Further additionally or alternately, the selectivity for 121° C.+ products can be 50 wt % or more, or 60 wt % or more, or 65 wt % or more, or 70 wt % or more, such as up to 90 wt % or possibly still higher.

In other aspects, a feed of C₃-C₈ olefins can be exposed to an MTW, MEL/MFI, MFS, MTT, and/or MFI framework catalyst to achieve a C₃-C₈ olefin conversion rate of 75 wt % or more, or 80 wt % or more, or 90 wt % or more, or 95 wt % or more, such as up to substantially complete conversion (100 wt %). Additionally, in some aspects, the conversion of C₄ olefins can be 80 wt % or more, or 90 wt % or more, such as up to substantially complete conversion of C₄ olefins (100 wt %). In such aspects, the conversion reaction can have a selectivity for formation of jet boiling range products of 40 wt % or more, or 50 wt % or more, or 60 wt % or more, or 70 wt % or more, such as up to 85 wt % or possibly still higher. Additionally or alternately, the selectivity for formation of heavy diesel products (that boil above the jet boiling range, or 300° C.-370° C.) can be reduced or minimized, so that the selectivity for heavy diesel products is 25 wt % or less, or 20 wt % or less. Further additionally or alternately, the selectivity for distillate products (121° C.-370° C.) can be 50 wt % or more, or 60 wt % or more, or 65 wt % or more, or 70 wt % or more, such as up to 90 wt % or possibly still higher. In such aspects, the conversion temperature can be 120° C. to 250° C., or 120° C. to 220° C., or 120° C. to 195° C., or 150° C. to 250° C., or 150° C. to 220° C., or 150° C. to 195° C.

In addition to high conversion rates for C₂ olefins and/or C₃-C₈ olefins, MTW, MEL/MFI, MFS, MTT, and/or MFI framework catalysts can have one or more additional unexpected benefits. For example, it has been discovered that catalysts having a MTW, MEL/MFI, and/or MFI framework can provide C₂ olefin conversion and/or C₃-C₈ olefin conversion with low formation of C₃-C₈ alkanes while having unexpectedly high yield of distillate boiling range components. As another example, it has been discovered that catalysts having a MEL, MFS, or MTT framework can provide elevated C₃-C₈ olefin conversion at low temperatures with an unexpectedly high yield of distillate boiling range components and an unexpectedly low yield of C₃-C₈ alkanes.

In addition to high conversion rates for both C₂ olefins and C₃-C₈ olefins, MOR, MTT, TON, FER, RFE and/or MFI framework catalysts can have one or more additional unexpected benefits. For example, in addition to high C₂ olefin conversion while providing substantial C₄ olefin conversion, it has been discovered that catalysts having a MOR, MTT, and/or TON framework can provide the high C₂ olefin conversion at unexpectedly low temperatures. As another example, in addition to high C₂ olefin conversion while providing substantial C₄ olefin conversion, it has been discovered that catalysts having a MTT, RFE, and/or MFI framework can provide the high C₂ olefin conversion in combination with unexpectedly low production of C₃-C₆ saturates. As still another example, in addition to high C₂ olefin conversion while providing substantial C₄ olefin conversion, it has been discovered that catalysts having a MOR and/or MFI framework can provide the high C₂ olefin conversion in combination with high selectivity for formation of jet boiling range products.

One way to characterize the conversion conditions for conversion of mixtures of olefins is by having a standardized condition to allow for comparison with other catalysts. In this discussion, one standardized condition that is used is a standardized condition for achieving 95 wt % conversion of C₂-C₆ olefins at a weight hourly space velocity (WHSV) of 2.0 hr⁻¹. The weight hourly space velocity is defined as the mass of the catalyst (mass) being exposed to a feed, divided by the flowrate of the feed (mass/time). Under these conditions, the temperature required to achieve 95 wt % conversion is referred to as a “corrected temperature for C₂-C₆ conversion”. Equation 1 provides a formula for determining the “corrected temperature for C₂-C₆ conversion” (Tcorr C₂-C₆) for achieved 95 wt % conversion of C₂-C₆ olefins based on conditions that are actually used.

$$T corr 95 \% ( ° C . ) = \frac{1}{( \frac{1}{( {T}_{a c t u a l} + 273 )} + ln ( \frac{ln ( 1 - {X}_{a c t u a l} ) * \frac{{WHSV}_{a c t u a l}}{2}}{ln ( 1 - 0.95 )} ) ) * \frac{R}{{E}_{a}}} - 273 \qquad ( 1 )$$

In Equation 1, the corrected temperature is calculated based on observed actual C₂-C₆ olefin conversion (X actual) at a measured temperature (T actual) and measured WHSV (WHSV actual), assuming a first order reaction, using the Arrhenius equation. The activation energy (Ea) for the conversion reaction for C₂-C₆ olefins is set at 45 KJ/mol based on prior empirical observations. R is the molar gas constant (R=8.314 J/mol*K). The corrected temperature is a proxy for catalyst activity as it expresses the temperature needed to reach a certain conversion level. Lower corrected temperature constitutes a more active catalyst.

Another standardized condition that is used is a standardized condition for achieving 95 wt % conversion of C₄ olefins at a weight hourly space velocity (WHSV) of 2.0 hr⁻¹. Under these conditions, the temperature required to achieve 95 wt % conversion is referred to as a “corrected temperature for C₄ conversion”. Equation 2 provides a formula for determining the “corrected temperature for C₄ conversion” (Tcorr C₄) based on conditions that are used.

$$T corr 95 \% C 4 ( ° C . ) = \frac{1}{( \frac{1}{( {T}_{a c t u a l} + 273 )} + ln ( \frac{ln ( 1 - {X}_{a c t u a l} ) * \frac{{WHSV}_{a c t u a l}}{2}}{ln ( 1 - 0.95 )} ) ) * \frac{R}{{E}_{a}}} - 273 \qquad ( 2 )$$

In Equation 2, The corrected temperature is calculated based on observed actual C₄ olefin conversion (X actual) at a measured temperature (T actual) and measured WHSV (WHSV actual), assuming a first order reaction, using the Arrhenius equation. The activation energy (Ea) for the conversion reaction for C₄ olefins is set at 90 KJ/mol. R is the molar gas constant (R=8.314 J/mol*K). The corrected temperature is a proxy for catalyst activity as it expresses the temperature needed to reach a certain conversion level. Lower corrected temperature constitutes a more active catalyst.

Although a pressure is not specified in either Equation 1 or Equation 2, it is generally understood that higher pressures tend to push reactions closer to equilibrium. It is believed that both Equation 1 and Equation 2 generally apply at pressures between 600 psia to 1200 psia (~4.2 MPa-a to ~8.3 MPa-a). If a specific pressure is desired for comparison purposes, a pressure of ~7.0 MPa-a can be used.

### Definitions

In this discussion, a zeolite is defined to refer to a crystalline material having a porous framework structure built from tetrahedra atoms connected by bridging oxygen atoms. Examples of known zeolite frameworks are given in the “Atlas of Zeolite Frameworks” published on behalf of the Structure Commission of the International Zeolite Association, 6th revised edition, Ch. Baerlocher, L. B. McCusker, D. H. Olson, eds., Elsevier, New York (2007) and the corresponding web site, http://www.iza-structure.org/databases/. In some aspects, a zeolite can correspond to an aluminosilicate having a zeolite framework structure. More generally, a zeolite can refer to aluminosilicates having a zeolite framework structure as well as zeolite framework structures containing oxides of atoms different from silicon and aluminum. Such oxides can include oxides of any other atoms generally known to be suitable for inclusion in a zeolite framework, such as oxides gallium, boron, germanium, phosphorus, zinc, and/or other transition metals that can substitute for silicon and/or aluminum in a zeolite framework. It is noted that under this definition, a zeolite can include materials such as silicoaluminophosphate (SAPO) materials or aluminophosphate (AIPO) materials.

It is noted that materials (such as ZSM-48) that previously were designated as *MRE framework in the Atlas of Zeolite Frameworks are now designated as RFE framework.

In this discussion, materials such as ZSM-11 correspond to intergrowth materials having both MEL and MFI framework. This is indicated in this discussion using MEL/MFI as the framework. In the Atlas of Zeolite Frameworks, ZSM-11 is identified as a MEL framework structure. As a practical matter, however, pure MEL framework structures can only be formed when the framework is 100% silica. For structures that have framework atoms other than silicon and oxygen, the material will be an intergrowth of MEL and MFI, with a MEL content of roughly 10% to 70% of the total amount of zeolite (or 15% to 50%, or 30% to 70%), with the balance corresponding to MFI.

In this discussion, pore channels of zeolitic framework structures can be referred to based on the number of heavy atoms (different from oxygen) that define the ring of the pore channel. Thus, a zeolitic framework structure that includes a 10-membered ring pore channel corresponds to a pore channel defined by rings that include 10 heavy atoms (such as Si and Al) plus 10 oxygen atoms.

In this discussion, references to a periodic table are defined as references to the current version of the IUPAC Periodic Table.

In this discussion, “noble metals” have the expected definition. Thus, noble metals are defined as Ru (Group 8), Os (Group 8), Rh (Group 9), Ir (Group 9), Pt (Group 10), Pd (Group 10), Au (Group 11), and Ag (Group 11). All other metals in Groups 5-14 are defined as non-noble metals. It is noted that Ga is a Group 13 metal.

In this discussion, characterization of the composition of feeds and conversion effluent is performed according to ASTM D5186.

In this discussion, reference is made to feeds that contain olefins within a carbon number range, such as a feed that contains C₃-C₈ olefins. It is understood that a feed that contains only C₃ olefins is considered a feed that contains C₃-C₈ olefins. Similarly, a feed that contains only C₄ olefins is considered a feed that contains C₃-C₈ olefins. In this discussion, a fraction that contains C₉-C₁₆ hydrocarbons is a fraction that can contain hydrocarbons having 9 to 16 carbons, but not hydrocarbons with less than 9 carbons or more than 16 carbons. A fraction that contains C₉-C₁₆+ hydrocarbons does not include hydrocarbons with less than 9 carbons, but can contain hydrocarbons that have more than 16 carbons.

In this discussion, the jet boiling range is defined as 121° C. to 300° C. A jet boiling range fraction is defined as a fraction with a T10 distillation point of 121° C. or higher, while also having a T90 distillation point and/or final boiling point of 300° C. or less. In this discussion, the diesel boiling range is defined as 149° C. to 300° C. A diesel boiling range fraction is defined as a fraction with a T10 distillation point of 149° C. or higher and a T90 distillation point of 370° C. or less. In this discussion, the heavy diesel boiling range is defined as 300° C. to 370° C. A heavy diesel boiling range fraction is defined as a fraction with a T10 distillation point of 300° C. or higher and a T90 distillation point of 370° C. or less. The distillate boiling range is defined as jet fuel plus diesel, which corresponds to 121° C.-370° C. A distillate boiling range fraction is defined as a fraction with a T10 distillation point of 121° C. or higher and a T90 distillation point of 370° C. or less. The naphtha boiling range is defined as C₅ (~29° C.) to 121° C. A naphtha boiling range fraction is defined as a fraction with a T10 distillation point of 29° C. or higher and a T90 distillation point of 121° C. or less. Fractional weight distillation values can be determined according to ASTM D2887. If ASTM D2887 is not suitable for some reason for a lower boiling fraction, ASTM D86 can be used instead.

For determination of olefin conversion and determination of yield of C₃-C₆ alkanes, gas chromatography can be used.

In this discussion, unless otherwise specified, conversion percentages correspond to single pass conversion percentages.

### Feedstock

In various aspects, a solid acid catalyst can be used for conversion of a feed containing a mixture of C₂ olefins and C₃-C₈ olefins, or for conversion of a feed containing C₃-C₈ olefins while having a reduced or minimized content of C₂ olefins.

In various aspects, the feed can have a total olefin content of 15 wt % to 100 wt %, or 25 wt % to 100 wt %, or 50 wt % to 100 wt %, or 70 wt % to 100 wt %, or 15 wt % to 70 wt %, or 25 wt % to 70 wt %, or 50 wt % to 70 wt %. Optionally but preferably, the feed can include 5.0 wt % or less of C₉₊ olefins relative to a weight of the feed, or 1.0 wt % or less, such as down to having substantially no content of C₉₊ olefins (less than 0.1 wt %). It is noted that the total olefin content of a feed corresponds to the total olefin content for the combination of a fresh feed and any optional recycle streams that may be included. If recycle is used to mitigate heat release during conversion, the resulting feed may have a lower olefin content, such as having 25 wt % or more of olefins.

In aspects where the feed contains a substantial portion of C₂ olefins, the feed can contain 5.0 wt % to 50 wt % of C₂ olefins relative to the weight of the feed, or 5.0 wt % to 35 wt %, or 5.0 wt % to 25 wt %, or 5.0 wt % to 15 wt %, or 10 wt % to 50 wt %, or 10 wt % to 35 wt %, or 10 wt % to 25 wt %. With regard to C₃-C₈ olefins, in such aspects the feed contains 10 wt % to 90 wt % of C₃-C₈ olefins relative to the weight of the feed, or 10 wt % to 70 wt %, or 10 wt % to 50 wt %, or 10 wt % to 30 wt %, or 20 wt % to 90 wt %, or 20 wt % to 70 wt %, or 20 wt % to 50 wt %. In such aspects, the feed contains 20 wt % or more of C₂-C₈ olefins, or 35 wt % or more, or 50 wt % or more, or 65 wt % or more, or 80 wt % or more, or 90 wt % or more, such as up to 100 wt %. A ratio of C₂ olefins to C₃-C₈ olefins can be from 0.1 to 3.0 (1:10 to 3:1), or 0.1 to 1.0, or 0.1 to 0.5, or 0.1 to 0.3, or 0.5 to 3.0, or 0.5 to 1.0. A ratio of C₃-C₅ olefins to C₂ olefins can be 1.0 to 10, or 2.0 to 10, or 3.0 to 10, or 4.0 to 10, or 1.0 to 7.0, or 2.0 to 7.0, or 3.0 to 7.0, or 4.0 to 7.0. A ratio of C₃ olefins to C₂ olefins can be 0.8 to 5.0, or 0.8 to 3.5, or 1.5 to 5.0, or 1.5 to 3.5, or 2.0 to 5.0, or 2.0 to 3.5.

In aspects where the feed contains a reduced or minimized content of C₂ olefins, the feed can contain 5.0 wt % or less of C₂ olefins relative to the weight of the feed, or 3.0 wt % or less, or 1.0 wt % or less, such as down to having substantially no C₂ olefin content. With regard to C₃-C₈ olefins, in such aspects the feed contains 10 wt % to 100 wt % of C₃-C₈ olefins relative to the weight of the feed, or 10 wt % to 85 wt %, or 10 wt % to 70 wt %, or 10 wt % to 50 wt %, or 10 wt % to 30 wt %, or 20 wt % to 100 wt %, or 20 wt % to 85 wt %, or 20 wt % to 70 wt %, or 20 wt % to 50 wt %, or 40 wt % to 100 wt %, or 40 wt % to 85 wt %. In such aspects, the feed can have a total olefin content of 15 wt % to 100 wt %, or 25 wt % to 100 wt %, or 50 wt % to 100 wt %, or 70 wt % to 100 wt %, or 15 wt % to 70 wt %, or 25 wt % to 70 wt %, or 50 wt % to 70 wt %. Optionally, in some aspects, the feed can contain a high percentage of C₃-C₈ olefins, corresponding to 85 wt % or more of the feed, or 90 wt % or more, such as up to the feed being substantially composed of C₃-C₈ olefins (100 wt %). In such optional aspects, the molar ratio of C₃-C₈ olefins to any other component in the feed can be 5.0 or more (i.e., 5:1), or 8.0 or more, or 10 or more, such as up to 500 (or possibly still higher as the C₃-C₈ olefin content in the feedstock approaches 100 wt %).

Depending on the aspect, the feedstock can also include other components, including but not limited to, isoparaffins, aromatics, impurities such as CO, and other components such as H₂ and/or n-alkanes. Optionally but preferably, the feed can include 5.0 wt % or less, relative to a weight of the feed, of C₉+ hydrocarbon that are different than olefins (paraffins, isoparaffins, aromatics, naphthenes), or 1.0 wt % or less, such as down to having substantially no content of C₉+ hydrocarbons that are different from olefins (less than 0.1 wt %).

It is noted that for molar ratios of one component to a second component as defined herein, if a feed is specified as having a molar ratio of a first component to a second component that is greater than a target value, such a feed is defined to satisfy that molar ratio when the second component is not present in the feed. For example, a statement that a feed has a molar ratio of C₃-C₈ olefins to isoparaffins of greater than 10 includes the situation where the feed does not contain any isoparaffins.

In some aspects, the C₃-C₈ olefins in the feed can primarily correspond to C₃-C₆ olefins. In such aspects, the weight ratio of C₃-C₆ olefins to C₇-C₈ olefins is 1.0 or higher, or 2.0 or higher, or 5.0 or higher, or 10 or higher, or 50 or higher, or 100 or higher, such as up to having substantially no content of C₇-C₈ olefins (weight ratio of 1000 or higher).

In some aspects, the feed can include 1.0 wt % or more of C₄ olefins, or 5.0 wt % or more, or 10 wt % or more, or 25 wt % or more, such as up to 100 wt %. In some aspects, the feed can include 5.0 wt % or more of C₃-C₄ olefins, or 10 wt % or more, or 25 wt % or more, or 40 wt % or more, or 60 wt % or more, or 80 wt % or more, such as up to 100 wt %.

In some aspects, the feed can have a molar ratio of C₃-C₈ olefins to isoparaffins of 0.1 or more, or 1.0, or 2.0 or more, or 5.0 or more, such as up to 100 or possibly still higher. In some aspects, the feed can have a molar ratio of C₃-C₆ olefins to isoparaffins of 0.1 or more, or 1.0, or 2.0 or more, or 5.0 or more, such as up to 100 or possibly still higher. Additionally or alternately, the feed can have a molar ratio of C₃-C₈ olefins to aromatics of 0.1 or more, or 1.0 or more, or 2.0 or more, or 5.0 or more, such as up to 50, or up to 100, or possibly still higher. Further additionally or alternately, the feed can have a molar ratio of C₃-C₆ olefins to aromatics of 0.1 or more, or 1.0 or more, or 2.0 or more, or 5.0 or more, such as up to 50, or up to 100, or possibly still higher. In this discussion, a feed that has a molar ratio of C₃-C₈ olefins (or C₃-C₆ olefins) to either isoparaffins or aromatics of 100 or more is defined as a feed that has substantially no content of isoparaffins/aromatics. (Thus, a feed that has ratio of C₃-C₆ olefins to isoparaffins/aromatics of 100 or more is defined to include feeds that contain C₃-C₆ olefins but no isoparaffins/aromatics, unless otherwise specified.)

The feed into the olefin conversion stage can optionally include one or more recycle streams in addition to fresh feed. When a recycle stream is included in the feed, the composition of the recycle stream can differ from the composition of the overall feed. For example, recycle streams are typically formed by separation of lower boiling components from the conversion effluent, such as by separating out a C₃-C₄ fraction, a C₃-C₅ fraction, or a C₃-C₆ fraction from the conversion effluent. These lower boiling fractions separated from the conversion effluent can include unreacted olefins that were not converted during a single pass through the conversion reactor. Recycling such olefins can improve the net conversion of the feed into products. However, such recycle fractions can also include paraffins and/or isoparaffins. Thus, addition of a recycle portion to a feed can result in a feed (fresh portion plus recycle portion) that will have a lower olefin content than the fresh portion alone. It is noted that distillate boiling range components are typically C₉+. Even though some C₇ or C₈ olefins may be present in the conversion effluent, generally the C₇-C₈ portion of the conversion effluent is used for another purpose. For example, one option is to use the C₇-C₈ fraction as a naphtha feed and/or naphtha blend component for gasoline formation.

In aspects where a recycle portion is included in the feed, the recycle portion can correspond to 1.0 vol % to 75 vol % of the feed to the conversion reaction, or 1.0 vol % to 50 vol %, or 1.0 vol % to 30 vol %, or 1.0 vol % to 10 vol %, or 10 vol % to 75 vol %, or 10 vol % to 50 vol %, or 10 vol % to 30 vol %, or 25 vol % to 75 vol %, or 25 vol % to 50 vol %.

In some aspects, the feed can correspond to a feed that includes CO as a contaminant. For example, some methanol to olefin processes can produce CO as a minor product along with ethylene. In various aspects, the feed can have a CO content of 100 wppm to 5000 wppm, or 100 wppm to 2500 wppm, or 100 wppm to 1000 wppm, or 250 wppm to 5000 wppm, or 250 wppm to 2500 wppm, or 250 wppm to 1000 wppm, or 500 wppm to 5000 wppm, or 500 wppm to 2500 wppm.

Additionally or alternately, other contaminants can also be present in the feed. The contaminants can correspond to one or more C₁-C₈ compounds that are not alkenes or alkanes. Examples of contaminants include alkynes (e.g., acetylene, methyl-acetylene), dienes (e.g., propadiene, butadienes, hexadienes), and oxygenates (ethers, aldehydes, and ketones). In various aspects, the feed can contain 1.0 wt % or less of oxygenates, including but not limited to dimethyl ether, methylethylether, acetaldehyde, acetone, methylacetate, and/or water.

In some aspects, the feed can include n-alkanes. In such aspects, the feed can also contain 1.0 wt % to 90 wt % of n-alkanes, or 1.0 wt % to 50 wt %, or 1.0 wt % to 20 wt %, or 10 wt % to 90 wt %, or 10 wt % to 50 wt %, or 30 wt % to 90 wt %.

In some aspects, the feed can include isoparaffins. In such aspects, the feed can also contain 1.0 wt % to 90 wt % of isoparaffins, or 1.0 wt % to 50 wt %, or 1.0 wt % to 20 wt %, or 10 wt % to 90 wt %, or 10 wt % to 50 wt %, or 30 wt % to 90 wt %.

In some aspects, the feed can include H₂. In such aspects, the ethylene-containing feed can contain 10 wt % or less of H₂, or 1.0 wt % or less, or 0.1 wt % or less, such as down to substantially no H₂ content (0.001 wt % or less).

### Examples of Configurations for Conversion

In the simplest configuration, a conversion process for conversion of C₂ and/or C₃-C₈ olefins can be performed without recycle. In this type of configuration, a feed containing C₂ and/or C₃-C₈ olefins is passed into a conversion reactor to generate a conversion effluent. The conversion effluent can then optionally be separated to recover a distillate boiling range product fraction and one or more other fractions. The one or more other fractions can include, but are not limited to, a diesel boiling range fraction, a heavy diesel boiling range fraction, a naphtha boiling range fraction, a light ends fraction (C₄), a C₃-C₆ fraction, a C₆-C₈ fraction, C₃-C₅ fraction, a C₅-C₈ fraction, a C₄-C₈ fraction, a C₃-C₈ fraction, a C₆-C₉ fraction, a C₅-C₉ fraction, and/or a C₄-C₉ fraction. It is noted that some of the fractions are complementary in aspects where a distillate (jet fuel plus diesel) boiling range fraction is formed. For example, if both a C₃-C₅ fraction and a distillate boiling range fraction are separated out from the conversion effluent, then a C₆-C₈ fraction or a C₆-C₉ fraction can also be formed.

In other aspects, one or more recycle loops can be included. Any convenient fraction that boils below the distillate boiling range can be used in part as a recycle fraction. For example, a C₃-C₈ fraction could be recycled, or a C₃-C₅ fraction, or a C₅-C₈ fraction, or a C₆-C₈ fraction, or a C₃-C₆ fraction, or a C₃-C₉ fraction, or a C₅-C₉ fraction, or a C₆-C₉ fraction, or any other convenient stream that may include C₃-C₆ olefins, C₃-C₄ olefins, and/or C₅-C₆ olefins. One way to form a recycle stream is to separate out a fraction corresponding to the desired components, and then remove a purge portion of the fraction from the system to prevent excessive build-up of n-paraffins.

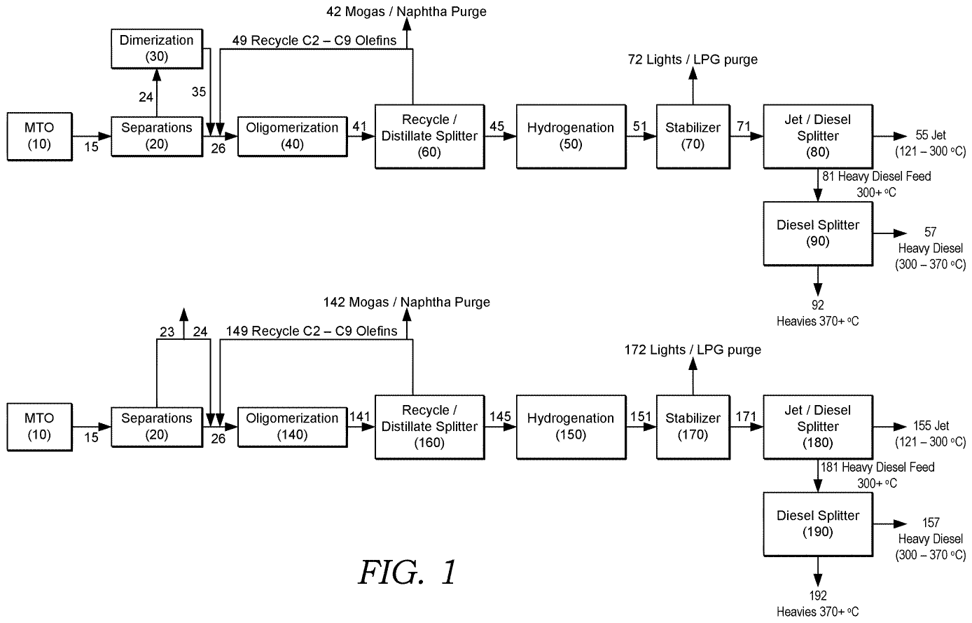

It is noted that, conventionally, the reduced ability of some conventional catalysts for conversion of C₂ olefins places a constraint on the types of reaction configurations that can be used. FIG. 1 shows a process overview of the difference between a conversion process with a catalyst that does not have sufficient activity for conversion of C₂ olefins (top configuration) versus a process (bottom configuration) using a catalyst as described herein. In the top configuration example in FIG. 1, a methanol-to-olefins unit 10 is used to supply olefins 15 for oligomerization. The olefins 15 are separated 20 into a primarily C₂ olefins stream 24 and a C₃₊ olefins stream 26. This separation allows for separate conversion of the C₂ olefins stream 24 in dimerization unit 30 to form a C₄₊ stream 35. Having a separate dimerization unit 30 allows the C₂ olefins to be converted using a catalyst and/or process that allows for efficient conversion. The C₄₊ stream 35 and the C₃₊ stream 26 are then oligomerized in oligomerization unit 40 to form an oligomerized product 41. The oligomerized product 41 is then separated 60 to into a lower boiling range product 42 and a higher boiling range product 45. Optionally, a recycle portion 49 of lower boiling range product 42 can be used as a recycle stream that is combined with C₄₊ stream 35 and C₃₊ stream 26 for oligomerization. The higher boiling range product is then hydrogenated 50 to form a hydrogenated product 51. The hydrogenated product 51 is then stabilized 70 to remove any C₄₋ products 72. Optionally a portion of the C₄₋ products can be used as liquefied propane gas (not shown). The stabilized hydrogenated product 71 is then passed into one or more separation stages, such as separation stage 80 and separation stage 90. In the example shown in the top portion of FIG. 1, separation stage 80 functions as a jet/diesel splitter, producing jet boiling range product 55 and heavy product 81. The separation stage 90 operates as a diesel splitter, allowing the heavy product 81 to be separated into a heavy diesel product 57 and a 370° C.+ product 92.

The bottom configuration example in FIG. 1 illustrates a simplified process flow that can be used with a catalyst that has sufficient activity for conversion of C₂ olefins and C₃₊ olefins in a single stage. In the bottom configuration, separation 20 to form C₂ olefins 24 and C₃₊ olefins 26 is optional. Performing the separation allows for blending of C₂ olefins and C₃₊ olefins in a desired ratio, but alternatively the olefins in effluent 15 could be used without further separation as the input to oligomerization 140. Oligomerization 140 forms an oligomerized product 141. The oligomerized product 141 can be separated 160 into a lower boiling range product 142 and a higher boiling range product 145. Optionally, a recycle portion 149 of lower boiling range product 142 can be used as a recycle stream that is combined with C₄₊ stream 35 and C₃₊ stream 26 for oligomerization. The higher boiling range product is then hydrogenated 150 to form a hydrogenated product 151. The hydrogenated product 151 is then stabilized 170 to remove any C₄₋ products 172. Optionally a portion of the C₄₋ products can be used as liquefied propane gas (not shown). The stabilized hydrogenated product 171 is then passed into one or more separation stages, such as separation stage 180 and separation stage 190. In the example shown in the bottom portion of FIG. 1, separation stage 180 functions as a jet/diesel splitter, producing jet boiling range product 155 and heavy product 181. The separation stage 190 operates as a diesel splitter, allowing the heavy product 181 to be separated into a heavy diesel product 157 and a 370° C.+ product 192.

The configurations shown in FIG. 1 include an optional recycle loop 49 or 149. In some aspects, a portion of the effluent generated by oligomerization 40 or 140 can be recycled as recycle stream 49 or 149. This optional recycle stream can then be added back at a convenient location, such as stream 26. The optional recycle portion can correspond to a portion that includes C₂-C₄ olefins. This can be a C₂-C₉ fraction, a C₂-C₈ fraction, a C₂-C₇ fraction, a C₂-C₅ fraction, or another convenient fraction that includes C₂-C₄ olefins. It is noted that the recycle fraction will typically include both olefins and paraffins corresponding to the specified type of fraction.

As another alternative, in either the top configuration or the bottom configuration of FIG. 1, a portion of C₂ stream 24 can be withdrawn as a separate product, such as stream 25 in the bottom configuration of FIG. 1. This can be useful for control of C₂ olefin and/or paraffin concentration in the system. This can also be useful for generating a separate C₂ olefin product.

As still another alternative, for the bottom configuration in FIG. 1, the olefin-containing effluent 15 can be passed directly into oligomerization 140 without performing separation 20. Conversion Conditions for Conversion of C₂ and/or C₃-C₈ olefins

In various aspects, a feed that contains C₂ olefins and/or C₃-C₈ olefins is exposed to a catalyst having a zeolitic framework under conversion conditions that include a conversion temperature of 120° C. to 300° C., or 120° C. to 250° C., or 120° C. to 220° C., or 120° C. to 195° C., or 150° C. to 300° C., or 150° C. to 250° C., or 150° C. to 220° C., or 150° C. to 195° C. In some aspects, the conversion conditions are selected to provide conversion of 75 wt % or more of the C₃-C₈ olefins in the feed, or 75 wt % or more of the C₃-C₆ olefins, or 80 wt % or more of the C₃-C₈ olefins in the feed, or 90 wt % or more, such as up to 100 wt %. Additionally or alternately, the conversion conditions can provide conversion of 80 wt % or more of the C₃-C₆ olefins, or 90 wt % or more of the C₃-C₆ olefins, such as up to 100 wt %. Further additionally or alternately, the conversion conditions are selected to provide conversion of 75 wt % or more of the C₄ olefins in the feed, or 80 wt % or more, or 90 wt % or more, such as up to 100 wt %. In this discussion, conversion of a feed component (such as conversion of C₃-C₈ olefins) is defined as the weight ratio of the weight of the feed component in the products relative to the weight of the feed component in the initial feed. For continuous processes, this weight comparison can be made based on an average weight per unit time (such as grams per minute) of the feed component in the product relative to the weight per unit time of the feed component in the feed. In aspects where the feed contains 5.0 wt % or more of C₂ olefins, in addition to conversion of C₃₊ olefins, the conversion conditions also can provide for conversion of 50 wt % to 100 wt % of the C₂ olefins, or 65 wt % to 100 wt %, or 80 wt % to 100 wt %, or 50 wt % to 90 wt %, or 65 wt % to 90 wt %, or 50 wt % to 80 wt %, or 65 wt % to 80 wt %.

In addition to a conversion temperature and an amount of conversion, the conversion conditions can further include a pressure of 0.1 MPa-a to 10 MPa-a, or 2.8 MPa-a to 10 MPa-a, or 5.0 MPa-a to 10 MPa-a, 0.1 MPa-a to 8.3 MPa-a, or 2.8 MPa-a to 8.3 MPa-a, or 5.0 MPa-a to 8.3 MPa-a, 0.1 MPa-a to 7.5 MPa-a, or 2.8 MPa-a to 7.5 MPa-a, or 5.0 MPa-a to 7.5 MPa-a. The conversion conditions can also include a weight hourly space velocity (WHSV) of 0.05 hr⁻¹ to 10 hr⁻¹, or 0.1 hr⁻¹ to 10 hr⁻¹, or 0.5 hr⁻¹ to 10 hr⁻¹, or 1.0 hr⁻¹ to 10 hr⁻¹, or 0.05 hr⁻¹ to 5.0 hr⁻¹, or 0.1 hr⁻¹ to 5.0 hr⁻¹, or 0.5 hr⁻¹ to 5.0 hr⁻¹, or 1.0 hr⁻¹ to 5.0 hr⁻¹.

In aspects where the catalyst in the conversion reactor is in the form of one or more fixed beds of catalyst, the temperature is defined as a weighted average bed temperature (WABT) for each catalyst bed. In this discussion, the weighted average bed temperature for a catalyst bed is defined as the average of the temperature of the feed at the top of the catalyst bed and the temperature of the effluent at the bottom of the catalyst bed. It is noted that as a run progresses, higher temperatures may be used to extend run length as the catalyst deactivates.

Regarding reactor temperature, a reactor can correspond to an adiabatic reactor or an isothermal reactor. An isothermal reactor is preferable, and simplifies determination of the bed temperature, as the entire reactor is substantially at the weighted average bed temperature. An example of an isothermal reactor is a multi-tubular packed bed reactor, using (raising) steam as a cooling medium and for heat integration with e.g. separations or other auxiliary unit operations. Adiabatic reactors require less capital expenditure, but the temperature can vary across the bed. For an adiabatic reactor, the weighted average bed temperature is used to characterize the reactor temperature.

This conversion is used to form a conversion effluent. The conversion effluent includes a distillate (jet fuel plus diesel) boiling range portion. In various aspects, the yield of 121° C.+ products (C₉₊ products), relative to the weight of total olefins in the feed, can be 40 wt % or more, or 50 wt % or more, or 60 wt % or more, such as up to 80 wt % or possibly still higher. It is noted that although the jet fuel boiling range is defined herein as 121° C. to 300° C., a “wide cut” jet fuel could potentially include components that boil at up to 330° C. Distillate (jet fuel plus diesel) includes components that boil up to 370° C. Additionally or alternately, the yield of C₉-C₁₆₊ products, relative to the weight of total olefins in the feed, can be 40 wt % or more, or 50 wt % or more, or 60 wt % or more, such as up to 80 wt % or possibly still higher. Further additionally or alternately, the yield of 121° C.-370° C. products can be 40 wt % or more, or 50 wt % or more, or 60 wt % or more, such as up to 80 wt % or possibly still higher. Still further additionally or alternately, the yield of 121° C.-300° C. products can be 40 wt % or more, or 50 wt % or more, or 60 wt % or more, such as up to 80 wt % or possibly still higher. Another option for characterizing the amount of conversion is to characterize the amount of C₄ conversion. In some aspects, the amount of conversion of C₄ olefins can be 75 wt % or more relative to the weight of C₄ olefins in the feed, or 80 wt % or more, or 90 wt % or more, or 95 wt % or more, such as up to 100 wt %.

Another option for characterizing the conversion conditions can be to specify the corresponding “corrected temperature” for the conversion conditions. The corrected temperature can correspond to a corrected temperature for conversion of C₂-C₆ olefins, or a corrected temperature for conversion of C₄ olefins. As defined above, the corrected temperature corresponds to the temperature for 95 wt % conversion of either the C₂-C₆ olefins or the C₄ olefins in the feed at a WHSV of 2.0 hr⁻¹ and a pressure of 650 psia, based on the values that were actually used. In some aspects, when using a MTW, MEL/MFI, MFS, MTT, and/or MFI framework catalyst for conversion of a feed containing C₂ olefins and/or C₃-C₈ olefins, where the olefins include C₄ olefins, the corrected temperature (C₄ olefins) for the conditions can be 150° C. to 220° C., or 150° C. to 200° C., or 170° C. to 220° C., or 170° C. to 200° C. In some aspects, when using a MTW, MEL/MFI, MFS, MTT, and/or MFI framework catalyst for conversion of a feed containing C₂ olefins and C₃-C₈ olefins, the corrected temperature (C₂-C₆ olefins) for the conditions can be 150° C. to 220° C., or 150° C. to 200° C., or 170° C. to 220° C., or 170° C. to 200° C. In still other aspects, when using a MOR, MTT, TON, FER, RFE and/or MFI framework catalyst for conversion of a feed containing C₂ olefins and C₃-C₈ olefins, the corrected temperature for the conditions can be 150° C. to 220° C., or 150° C. to 200° C., or 170° C. to 220° C., or 170° C. to 200° C. More generally, when using a solid acid catalyst, the corrected temperature for conversion of C₂-C₆ olefins can be 150° C. to 220° C., or 150° C. to 200° C., or 170° C. to 220° C., or 170° C. to 200° C.

An additional advantage of performing conversion of C₂ and/or C₃-C₈ olefins using some types of solid acid catalysts, such as MTW, MEL/MFI, MFS, MTT, MOR, TON, FER, RFE and/or MFI framework catalysts, is that a high yield of distillate boiling range products can be achieved while reducing or minimizing production of diesel boiling range products. In some aspects, the yield of diesel boiling range products is 25 wt % or less, or 20 wt % or less, or 15 wt % or less, or 10 wt % or less, such as down to 0.1 wt % or possibly still lower.

Still another advantage of performing conversion of C₂ and/or C₃-C₈ olefins in the presence of some types of solid acid catalysts, such as MTW, MEL/MFI, MFS, MTT, MOR, TON, FER, RFE and/or MFI framework catalysts, is that the yield of light (C₃-C₈)alkanes can be reduced or minimized. In some aspects, the yield of C₃-C₆ alkanes is 4.0 wt % or less, or 3.0 wt % or less, or 2.0 wt % or less, or 1.0 wt % or less, such as down to 0.1 wt % or possibly still lower. It is noted that a reduced or minimized yield of C₃-C₆ alkanes can be used as a proxy for a reduced or minimized yield of C₃-C₈ alkanes.

### Solid Acid Catalysts

In various aspects, conversion of feed containing C₂ olefins and/or C₃-C₈ olefins to form a conversion effluent can be performed as in a single stage process in the presence of a solid acid catalyst, such as a MTW, MEL/MFI, MFS, MTT, MOR, TON, FER, RFE, and/or MFI framework catalyst (i.e., a catalyst containing a zeolite having a MTW, MEL/MFI, MFS, MTT, MOR, TON, FER, RFE, and/or MFI framework). Such solid acid catalysts can provide unexpected benefits for conversion of a feed containing C₂ olefins and/or C₃-C₈ olefins due to the catalysts having a framework structure that includes one or more of beneficial features A)-E).

A) One example of a beneficial feature is having a 2-dimensional (2-D) or 3-dimensional (3-D) zeolite framework structure that has a small elliptical 8-member ring channel connected to a 10-member ring or other moderately larger channel. This type of feature can be beneficial for improved conversion of C₂ olefins. Without being bound by any particular theory, a zeolitic framework structure that includes a small elliptical 8-member ring channel can be large enough to allow C₂ olefins to enter the pore channel. When the 8-member ring channel has connectivity to a moderately larger second pore channel, the combination of the 8-member ring channel and the moderately larger channel can provide a confinement effect that improves the rate of oligomerization to larger olefins while still allowing C₈₊ olefins to exit from the framework structure. MOR and FER are examples of framework structures that include this type of combination of an 8-member ring channel connected to a moderately larger pore channel. Other framework structures with this type of beneficial feature include framework structures that include an elliptical 8-member ring pore channel with dimensions of 2.2-3.5 Angstroms (minor axis) by 4.0 to 6.0 Angstroms (major axis). The moderately larger framework structure can be a 10-member ring pore channel, as well as selected 12-member ring or 14-member ring pore channels. MOR is an example of a framework structure with a sufficiently small 12-member ring channel. It is noted that many zeolites include 8-member ring pore channels that are connected to 12-member ring or 14-member ring cages that are too large to provide the confinement benefit for the C₂ olefins. In this discussion, cages consist of 12-member rings or 14-member rings, but are limited by a smaller window, such as 8-member ring window. In this discussion, for channels, the size of the window and channel are the same, such as having both 12-member ring windows and 12-member ring channels, or having both 14-member ring windows and 14-member ring channels. Examples of framework structures where the 12-member ring or 14-member ring cages are too large to provide the confinement effect include LTA, DDR, CHA, AFX, and AEI. It is further noted that when a zeolitic framework structure contains only 8-member rings as the largest pore channels—whether with small channels/cages (such as GIS, CDO, or EAB) or large cages (such as CHA or LTA)—the oligomerization products that correspond to C₈ or larger olefins (C₈₊) are too bulky to diffuse through the 8-member ring windows. Consequently, such frameworks are unsuitable for oligomerization applications.

B) Another example of a beneficial feature is having a pore channel in the framework structure that provides effective confinement for C₂ olefins, but with pore channels that are too narrow for effective fast growth of C₄ olefin monomers. This type of feature can be beneficial for conversion of C₂ olefins. Zeolites that include a 1-dimensional (1-D) 10-member ring pore channel are examples of this type of framework structure. More generally, framework structures having a 1-D pore channel where the maximum sphere diameter that can be included in the pore channel is 4.0 Angstroms to 6.0 Angstroms are examples of this type of framework structure. Specific examples of such 1D 10-member ring pore channel structures include TON (ZSM-22), MTT (ZSM-23, SSZ-32, SSZ-32x), and MTT/TON intergrowth. Other examples of such framework structures include RFE (ZSM-48) and AEL (SAPO-11). It is noted that MOR framework structure includes unidirectional 12-membered ring channels measuring approximately 7.0 Angstroms by 6.5 Angstroms. MOR also includes smaller 8-membered ring channels with elliptical openings (5.7 Angstroms by 2.6 Angstroms) that run parallel to and connect to the 12-membered ring channels. This results in the MOR framework structure forming side pockets with 8-membered ring windows of size 4.8 Angstroms by 3.4 Angstroms. This means that MOR provides two distinct channel types that exhibit substantially distinct reaction behaviors.

Additional examples of framework structures that include feature A and/or feature B are CSV, EON, HEU, IFW, SFO, YFI, and IWW.

C) Still another example of a beneficial feature is having a framework structure containing either 1-D or 3-D pore channels with a maximum diameter of a sphere that can be included in a pore channel of 5.0 to 6.5 Angstroms. This primarily corresponds to framework structures with 1-D or 3-D 10-membered ring pore channels, with the exception that MTW provides a 1-D 12-membered ring pore channel having this pore channel size. This type of feature is beneficial for reducing or minimizing the formation of C₃-C₆ alkanes. Examples of 1-D 10-membered ring framework structures having a maximum sphere diameter that can be included in a pore channel of 5.0 Angstroms to 6.5 Angstroms include TON (ZSM-22), MTT (ZSM-23, SSZ-32, SSZ-32x), intergrown TON/MTT, ZSM-48 (RFE), AEL (SAPO-11), and UZM-55 (intergrowth of ZSM-12 & ZSM-48). Examples of 3-D 10-membered ring framework structures having a maximum sphere diameter that can be included in a pore channel of 5.0 to 6.5 Angstroms include MFI (ZSM-5), MFI/MEL intergrowth, MEL, and -SVR. It is further noted that MFS (ZSM-57) is a 2-D 10-membered ring framework structure that can provide reduced or minimized formation of small alkanes. Generally, 2-D 10-membered ring framework structures can include a maximum diameter sphere size of 7.0 Angstroms or less. However, MFS framework structure results in crystals with a plate-like morphology having a thickness of 50 nm or less. Without being bound by any particular theory, it is believed that this morphology allows diffusion through the 10-membered ring channels across the entire thickness of the plate, thus reducing the residence time of oligomerization products within the pore channels. This is believed to result in reduced production of small alkanes, as well as increased selectivity for formation of C₈ and C₉ jet boiling range compounds. The rapid diffusion of C₈-C₉ oligomers out of the pores prevents further oligomerization or coking.

(D) Yet other examples of beneficial features for reducing or minimizing production of small alkanes can be having a suitable particle size for the solid acid catalyst and/or a suitable diffusion length within the framework structure. In this discussion, particle size refers to the largest dimension of a particle, which typically corresponds to a length (or could correspond to a diameter, for a roughly spherical particle or a thin disk particle). Diffusion path length refers to the length of the largest diameter channel. For example, if a particle contains both 12-member ring channels and 8-member ring channels, the diffusion path length for the particle would be defined based on the path length of the 12-member ring channel.

In some aspects, a solid acid catalyst can have a particle size of 500 nm or less, or 300 nm or less, or 100 nm or less, such as down to 20 nm or possibly still smaller. Additionally or alternately, a solid acid catalyst can have a maximum diffusion length within the framework structure of 500 nm or less, or 300 nm or less, or 100 nm or less, such as down to 20 nm or possibly still smaller.

It is noted that when the diffusion path is parallel to the length of a particle, the effects from diffusion path length and particle size can be correlated. For example, in zeolites that form needles with diffusion paths that run the length of the needle, it can be valuable to reduce or minimize the length of the needle. For a zeolite such as ZSM-57 (MFS), where the diffusion path is perpendicular to the length (and in this case also the width) of a particle, it can be valuable to reduce or minimize the thickness.

Additional examples of framework structures that include feature C and/or feature D are ITH, ITR, and SZR.

(E) Still another example of a beneficial feature can be having a relatively low Si to Al molar ratio in the framework structure. In some aspects, having a relatively low molar ratio of Si to Al in a zeolitic framework structure can contribute to increased conversion of C₂ olefins and/or decreased light alkane production. In such aspects, the molar ratio of Si to Al in a zeolitic framework structure can be 5 to 40, or 5 to 25, or 8 to 40, or 8 to 25.

Examples of crystalline materials (zeolites) of the MTW framework type include, but are not limited to, ZSM-12, NU-13, Theta-3, CZH-5, TPZ-12, and any combination thereof. In some aspects, the MTW framework catalyst can correspond to ZSM-12. In some aspects, an MTW framework catalyst can be an aluminosilicate material having a silica to alumina molar ratio of 20 or more, such as from 30 to 50. In other aspects, a MTW framework structure catalyst can have a framework structure that includes one or more atoms different from silicon, aluminum, and oxygen.

Examples of crystalline materials (zeolites) of the MTT framework type include, but are not limited to, ZSM-23, KZ-1, ISI-4, EU-13, and any combination thereof. In some aspects, the MTT framework catalyst can correspond to ZSM-23. In some aspects, an MTT framework catalyst can be an aluminosilicate material having a silica to alumina molar ratio of 20 or more, such as from 30 to 50. In other aspects, a MTT framework structure catalyst can have a framework structure that includes one or more atoms different from silicon, aluminum, and oxygen.

Examples of crystalline materials (zeolites) of the MEL/MFI framework type include, but are not limited to, ZSM-11, EMM-30, Boralite D, and any combination thereof. In some aspects, the MELMFI/framework catalyst can correspond to ZSM-11, EMM-30, or a combination thereof. In some aspects, an MEL/MFI framework catalyst can be an aluminosilicate material having a silica to alumina molar ratio of 20 or more, such as from 25 to 50. In other aspects, a MEL/MFI framework structure catalyst can have a framework structure that includes one or more atoms different from silicon, aluminum, and oxygen.

Examples of crystalline materials (zeolites) of the MFS framework type include, but are not limited to, ZSM-57, COK-5, and any combination thereof. In some aspects, the MFS framework catalyst can correspond to ZSM-57. In some aspects, an MFS framework catalyst can be an aluminosilicate material having a silica to alumina molar ratio of 30 or more, such as from 40 to 60. In other aspects, a MFS framework structure catalyst can have a framework structure that includes one or more atoms different from silicon, aluminum, and oxygen.

Examples of crystalline materials (zeolites) of the MOR framework type include, but are not limited to, mordenite, Ca-Q, Na-D, LZ-211, EMM-34 and any combination thereof. In some aspects, the MOR framework catalyst can correspond to mordenite. In some aspects, an MOR framework catalyst can be an aluminosilicate material having a silica to alumina molar ratio of 10 or more, such as from 12 to 25. In other aspects, a MOR framework structure catalyst can have a framework structure that includes one or more atoms different from silicon, aluminum, and oxygen.

Examples of crystalline materials (zeolites) of the TON framework type include, but are not limited to, Theta-1, KZ-2, NU-10, ZSM-22, ISI-1, and any combination thereof. In some aspects, the TON framework catalyst can correspond to ZSM-22, Theta-1, or a combination thereof. In some aspects, an TON framework catalyst can be an aluminosilicate material having a silica to alumina molar ratio of 30 or more, such as from 40 to 90. In other aspects, a TON framework structure catalyst can have a framework structure that includes one or more atoms different from silicon, aluminum, and oxygen.

Examples of crystalline materials (zeolites) of the FER framework type include, but are not limited to, ZSM-35, ferrierite, NU-23, FU-9, ISI-6, and any combination thereof. In some aspects, the FER framework catalyst can correspond to ZSM-35, ferrierite, or a combination thereof. In some aspects, an FER framework catalyst can be an aluminosilicate material having a silica to alumina molar ratio of 10 or more, such as from 10 to 50. In other aspects, a FER framework structure catalyst can have a framework structure that includes one or more atoms different from silicon, aluminum, and oxygen.

An example of crystalline materials (zeolites) of the RFE framework type is ZSM-48, which has orthorhombic or pseudo-orthorhombic symmetry and ten-ring non-interconnecting, linear channels whose ideal dimensions are 5.5×5.6 Å. The framework structure of ZSM-48 has been assigned the three-letter code RFE. RFE framework type can include (but is not limited to) zeolites of the ZSM-48 family such as at least one of COK-8, EU-2, EU-11, IZM-1, SSZ-91, ZBM-30, RUB-58 and ZSM-48.

Examples of crystalline materials (zeolites) of the MFI framework type include, but are not limited to, ZSM-5, NU-5, FZ-1, AZ-1, LZ-105, EMM-20, and any combination thereof. In some aspects, the MFI framework catalyst can correspond to ZSM-5. In some aspects, the MFI framework catalyst can correspond to EMM-20. In some aspects, an MFI framework catalyst can be an aluminosilicate material having a silica to alumina molar ratio of 20 or more, such as from 25 to 50. In other aspects, a MFI framework structure catalyst can have a framework structure that includes one or more atoms different from silicon, aluminum, and oxygen.

In various aspects, a catalyst and/or catalyst composition containing a zeolite having a MTW, MEL/MFI, MFS, MTT, MOR, TON, FER, RFE, and/or MFI framework, and/or having one or more of beneficial features A)-E), can be substantially free of catalytic metals supported on the catalyst. The amount of catalytic metal on a catalyst that corresponds to “substantially free” can vary depending on the nature of the metal. In some aspects, the catalyst and/or catalyst composition can have less than 0.75 wt % of Group 5-10 non-noble metals supported on the catalyst and/or catalyst composition, or 0.5 wt % or less, or 0.1 wt % or less, such as down to having substantially no supported Group 5-10 metals (0.01 wt % and/or detection limit), relative to a weight of the catalyst and/or catalyst composition. It is noted that this means that the combined amount of all Group 5-10 non-noble metals is less than the specified amount (i.e., less than 0.75 wt %, or 0.5 wt % or less, or 0.1 wt % or less). Additionally or alternately, the catalyst and/or catalyst composition can have less than 0.75 wt % of Group 5-10 and Group 13 non-noble metals, or 0.5 wt % or less, or 0.1 wt % or less, such as down to having substantially no supported Group 5-10 and Group 13 metals (0.01 wt % and/or detection limit). Further additionally or alternately, the catalyst and/or catalyst composition can have less than 0.75 wt % of Group 5-14 metals supported on the catalyst and/or catalyst composition, or 0.5 wt % or less, or 0.1 wt % or less, such as down to having substantially no supported Group 5-14 metals (0.001 wt % and/or detection limit), relative to a weight of the catalyst and/or catalyst composition. It is explicitly defined herein that a catalyst that is described as containing, including, or comprising less than a certain amount of a supported metal includes the situation where the catalyst includes none of the supported metal.

In some aspects, the catalyst and/or catalyst composition can have 0.05 wt % or less of Group 8-11 noble metals supported on the catalyst and/or catalyst composition, or 0.01 wt % or less, such as down to having substantially no Group 8-11 noble metals (0.001 wt % and/or detection limit). Additionally or alternately, the catalyst and/or catalyst composition can have 0.05 wt % or less of Pt, Pd, or a combination thereof, or 0.01 wt % or less, such as down to having substantially no Pt, Pd, or a combination thereof (0.001 wt % and/or detection limit).

In some aspects, catalysts and catalyst compositions described herein can include a binder. In other aspects, catalysts and catalyst compositions disclosed herein can include “self-bound” compositions that contain no, or substantially no, binder or additives. Self-bound catalysts (alternatively referred to as unbound or binder-free catalysts), are catalysts that do not contain a separately added matrix or binder material. The method described herein enables extrudates having high crush strength to be produced on conventional extrusion equipment and accordingly, the method is eminently suitable for producing the high activity self-bound catalysts. In some aspects, catalysts can “consist essentially of” zeolite, and include no binder or additives and containing only unavoidable levels of impurities or non-active substances. Zeolite catalysts may be contaminated with other crystalline materials in some embodiments, such as ferrierite or quartz. These contaminants may be present in quantities of 10 wt % or less, such as 5.0 wt % or less.

In aspects where the catalyst composition includes a binder, the binder materials may include inorganic oxides, such as alumina, silica, titania, zirconia, and mixtures and compounds thereof, may be present in the catalyst in amounts 60 wt % or less, for example 50 wt % or less, such as 40 wt % or less, for example 30 wt % or less, such as 20 wt % or less, such as down to 1.0 wt % or possibly still lower. Where a non-alumina binder is present, the amount employed may be as little as 1.0 wt %, or 5.0 wt % or more, for example 10 wt % or more.

In aspects where the catalyst composition includes a binder, the catalyst composition can include a zeolite framework structure material in an amount of 30 wt % or greater, or 40 wt % or greater, or 50 wt % or greater, or 60 wt % or greater, or 70 wt % or greater, or 80 wt % or greater, or 90 wt % or greater, such as up to 99 wt % or possibly still higher. For example, the amount of zeolite framework structure material can be from 40 wt % to 99.99 wt %, from 50 wt % to 99.95 wt %, from 60 wt % to 99.9 wt %, from 70 wt % to 99.8 wt %, or from 80 wt % to 99.7 wt %.

The catalysts can be formed by any convenient method, including conventional extrusion, ion exchange, and calcination methods.

### Additional Catalysts

In some aspects, a conversion reaction stage can include a catalyst containing a zeolite having one or more of beneficial features A)-E) and at least one additional catalyst. This can correspond to a catalyst system, where multiple types of catalyst particles are used, and/or this can correspond to a catalyst where an additional zeolite framework structure is present within a single type of catalyst particle.

In aspects where at least one additional zeolite framework structure is present in the conversion reaction stage, any convenient type of zeolite framework with activity for oligomerization and/or conversion can be used as the at least one additional zeolite framework structure material. In some aspects, the multiple framework structures correspond to multiple framework structures that each have one or more of beneficial features A)-E) (such as MTW, MEL/MFI, MFS, MTT, MOR, TON, FER, RFE, and/or MFI framework structures). Examples of other zeolite framework structures that can be used for oligomerization include, but are not limited to, BEA (e.g., zeolite Beta), FAU (e.g., zeolite Y), MOR, RFE, FER, CHA (e.g., SAPO-34), and combinations thereof.

When additional catalyst particles are present that include at least one additional zeolite framework structure, in some aspects the additional catalyst particles can be substantially free of supported metals as described herein, such as being substantially free of Group 5-10 and Group 13 non-noble metals, and/or substantially free of Group 8-11 noble metals, and/or substantially free of Group 5-14 metals (non-noble and/or noble). In other aspects, the additional catalyst particles can include a supported metal.

In aspects where the additional catalyst particles include a supported metal, one option is to use a supported metal that corresponds to a hydrogenation catalyst. Examples of hydrogenation catalysts can include noble metals, such as Pd, Pt, Rh, Ru, Ir, Os, Ag, Au; or non-noble metals, such as Mo, Co, Ni, Fe; or any combination thereof, such as the combination of two noble metals, two non-noble metals, or a combination of noble and non-noble metals.

When one metal is present, the amount of the one metal can be 0.01 wt % to 5.0 wt %, based on the total weight of the additional catalyst particle, or 0.1 wt % to 5.0 wt %, or 0.5 wt % to 5.0 wt %, or 1.0 wt % to 5.0 wt %, or 0.01 wt % to 3.5 wt %, or 0.1 wt % to 3.5 wt %, or 0.5 wt % to 3.5 wt %, or 1.0 wt % to 3.5 wt %, or 0.01 wt % to 1.5 wt %, or 0.1 wt % to 1.5 wt %, or 0.5 wt % to 1.5 wt %.

When more than one hydrogenation metal is present, the collective amount of hydrogenation metals can be 0.01 wt % to 5.0 wt %, based on the total weight of the additional catalyst particle, or 0.1 wt % to 5.0 wt %, or 0.5 wt % to 5.0 wt %, or 1.0 wt % to 5.0 wt %, or 0.01 wt % to 3.5 wt %, or 0.1 wt % to 3.5 wt %, or 0.5 wt % to 3.5 wt %, or 1.0 wt % to 3.5 wt %, or 0.01 wt % to 1.5 wt %, or 0.1 wt % to 1.5 wt %, or 0.5 wt % to 1.5 wt %.

In aspects where both Pt and Pd are present as the hydrogenation metals, the molar ratio of Pt to Pd can be from 1:3 to 4:1, or from 1:4 to 3:1, or from 1:2 to 4:1, or from 1:2 to 3:1. The amounts of metal(s) may be measured by methods specified by ASTM for individual metals, including but not limited to, atomic absorption spectroscopy (AAS).

In some aspects, the additional catalyst particles can also include a support material or binder. Examples of suitable support materials and/or binders may include clay, alumina, silica, titania, zirconia, aluminosilicates, zeolites, carbon, and combination thereof. In some aspects, the silica support can be an amorphous silica support. In some aspects, the support can be a mesoporous crystalline or semi-crystalline support material. Examples of mesoporous silica materials suitable for use as a support can include, but are not limited to, zeolites, such as MCM-41, other M41S structures, SBA-15, and the like, and any combination thereof.

In aspects where the at least one additional catalyst is provided by having a catalyst system including multiple types of catalyst, the at least one additional catalyst and the catalyst having one or more of beneficial features A)-E) (such as an MTW, MEL/MFI, MFS, MTT, MOR, TON, FER, RFE, and/or MFI framework structure) can be arranged in any convenient manner. In some aspects, the catalyst having the one or more beneficial features and the at least one additional catalyst can correspond to a physical mixture of catalyst particles. Additionally or alternately, in some aspects a stacked bed arrangement can be used, so that the feed is contacted first with one type of catalyst particle (such as in one or more catalyst beds), followed by contacting the feed with another type of catalyst particle (such as in one or more additional catalyst beds, or in a downstream portion of a catalyst bed containing the first type of catalyst). In such aspects, a feed can be contacted first with the catalyst having the one or more beneficial features, or the feed can be contacted first with an additional catalyst.

Still another option can be to have a catalyst that is formulated so that the framework structure providing the one or more beneficial features and at least one additional zeolite framework structure are incorporated into a single catalyst particle.

In aspects where at least one additional catalyst is included in the conversion stage, any convenient amount of the catalyst having one or more beneficial features (such as MTW, MEL/MFI, MFS, MTT, MOR, TON, FER, RFE, and/or MFI framework structure) and the at least one additional catalyst can be used. In some aspects, a weight ratio of the catalyst particles having the one or more beneficial features to the particles of the at least one additional catalyst can be 0.2 to 10, or 0.33 to 10, or 0.5 to 10, or 1.0 to 10, or 0.2 to 5.0, or 0.33 to 5.0, or 0.5 to 5.0, or 1.0 to 5.0.

### Integrated Conversion of Oxygenates to Distillate Boiling Range Products

Olefin oligomerization can be performed as part of an integrated process for conversion of oxygenates to distillate boiling range products. As an example, alcohols such as methanol and/or ethanol can be converted to olefins. The olefins can then be oligomerized to form products such as distillate boiling range products. Preferably, the oxygenate conversion process can be designed to preferentially form olefins that are beneficial for oligomerization processes as described herein.

Suitable and/or effective conditions for performing a conversion reaction for conversion of alcohols (such as methanol and/or ethanol) to olefins may include average reaction temperatures of 200° C. to 550° C. (or 250° C. to 550° C., or 300° C. to 550° C., or 350° C. to 550° C., or 400° C. to 500° C.), total pressures between 10 psig (70 kPa-g) to 400 psig (2700 kPa-g), or 50 psig (350 kPa-g) to 350 psig (2400 kPa-g), or 100 psig 0700 kPa-g) to 300 psig (2100 kPa-g), and an alcohol space velocity between 0.1 h⁻¹ to 10 h⁻¹ based on weight of alcohol relative to weight of catalyst. For example, the average reaction temperature may be at least 200° C., or at least 250° C., or at least 300° C., or at least 350° C., or at least 400° C., or at least 450° C. Additionally or alternately, the average reaction temperature can be 550° C. or less, or 500° C. or less, or 450° C. or less, or 400° C. or less. In this specification, average reaction temperature is defined as the average of the temperature at the reactor inlet and the temperature at the reactor outlet for the reactor where the conversion reaction is performed. In some aspects, where lower pressures are used, the pressure can correspond to 70 kPa-g to 700 kPa-g. As another example, the total pressure can be at least 70 kPa-g, or at least 350 kPa-g, or at least 500 kPa-g, or at least 700 kPa-g, or at least 1000 kPa-g. Additionally or alternately, the total pressure can be 3000 kPa-g or less, or 2700 kPa-g or less, or 2400 kPa-g or less, or 2100 kPa-g or less.

A variety of zeolite catalysts can be suitable as a conversion catalyst and/or an oligomerization catalyst. The conversion catalyst may comprise a zeolite in its original crystalline form or after formulation into catalyst extrudates, such as by extrusion. When a formulated catalyst is used, the formulated catalyst can include a binder, or can be formulated without a binder.

For catalysts that include a binder, zeolite crystals can be combined with a binder, such as, for example, one or more of Al₂O₃, TiO₂, ZrO₂, SiO₂, SiO₂/Al₂O₃, and MgO to form bound catalysts. Generally, a binder can be present in an amount between 1 wt % to 90 wt % relative to a weight of the catalyst, or 10 wt % to 90 wt %, or 20 wt % to 90 wt %, or 1 wt % to 70 wt %, or 10 wt % to 70 wt % or 20 wt % to 70 wt %, or 1 wt % to 40 wt %, or 10 wt % to 40 wt %, or 20 wt % to 40 wt %. Combining the zeolite and the binder can generally be achieved, for example, by mulling a mixture of the zeolite and binder (optionally an aqueous mixture) and then extruding the mixture into catalyst pellets.

An option for characterizing a zeolite or other molecular sieve is based on the nature of the ring channels in the zeolite. The ring channels in a zeolite framework structure can be defined based on the number of atoms including in the ring structure that forms the channel. In some embodiments, a zeolite framework structure can include at least one ring channel based on a 10-member ring. In such aspects, the framework structure preferably does not have any ring channels based on a ring larger than a 10-member ring. Examples of suitable framework structures having a 10-member ring channel but not having a larger size ring channel include EUO, FER, IMF, LAU, MEL, MFI, MEL/MFI, MFS, MTT, MWW, NES, PON, SFG, STF, STI, TON, TUN, MRE, and PON framework types.

In some alternative embodiments, the zeolite can be a molecular sieve that includes an 8-member ring channel (small pore molecular sieves), a 10-member ring channel (as described above), or a 12-member ring channel (large pore molecular sieves), but does not have any ring channels based on a ring larger than a 12-member ring. In such aspects, suitable large pore molecular sieves can include those having AFI, AFS, ATO, ATS, *BEA, BEC, BOG, BPH, CAN, CON, EMT, EON, EZT, FAU, GME, GON, IFR, ISV, -*ITN, IWR, IWW, LTL, MAZ, MEI, MOR, MOZ, MSE, MTW, OFF, OKO, OSI, SAF, SAO, SEW, SFE, SFO, SSF, SSY, and USI framework types. In such aspects, suitable small pore molecular sieves can include those having the AEI, AFT, AFX, ATT, DDR, EAB, EPI, ERI, KFI, LEV, LTA, MER, MON, MTF, PAU, PHI, RHO, and SFW framework types.

Generally, a zeolite having the desired activity can have a silicon to aluminum molar ratio of 10 to 300, or 15 to 100, or 20 to 80, or 20 to 40. In some aspects, the silicon to aluminum ratio can be at least 20, or at least 30, or at least 40. In such embodiments, the silicon to aluminum ratio can optionally be 100 or less, or 80 or less, or 60 or less, or 50 or less, or 40 or less. Typically, reducing the silicon to aluminum ratio in a zeolite can result in a zeolite with a higher acidity, and therefore higher activity for cracking of hydrocarbon or hydrocarbonaceous feeds, such as petroleum feeds. With respect to conversion of alcohols and/or ethers to olefins, such increased cracking activity due to a decrease in the silicon to aluminum ratio may result in increased formation of residual carbon or coke during the conversion reaction. Such residual carbon can deposit on the zeolite conversion catalyst, leading to a change in the properties of the catalyst over time. Having a silicon to aluminum ratio of at least 40, or at least 50, or at least 60, can reduce/minimize the amount of additional residual carbon formed due to the acidic or cracking activity of the conversion catalyst.

In some aspects, the conversion catalyst can include and/or be enhanced by one or more metals selected from groups 1 to 14 of the periodic table. The metal can be incorporated into the zeolite by any convenient method known in the art, such as by impregnation or by ion exchange. The amount of metal can be expressed as a weight percentage of the conversion catalyst, such as having at least about 0.1 wt. % of metal, at least about 0.25 wt. %, at least about 0.5 wt. %, at least about 0.75 wt. %, or at least about 1.0 wt. %. Additionally, or alternatively, the amount of metal can be about 20 wt. % or less, for example about 10 wt. % or less, about 5 wt. % or less, about 2.0 wt. % or less, about 1.5 wt. % or less, about 1.2 wt. % or less, about 1.1 wt. % or less, or about 1.0 wt. % or less.

In some aspects, the conversion catalyst can include one or more metals from groups 12 to 14 of the periodic table and thus include the metal zinc. In additional or alternate embodiments, the conversion catalyst can include one or more metals from groups 1 and 2 of the periodic table. The total weight of the metals can be 0.1 wt % to 10 wt % based on the total weight of the conversion catalyst, or 0.1 wt % to 5.0 wt %, or 0.1 wt % to 2.0 wt %, or 0.5 wt % to 10 wt %, or 0.5 wt % to 5.0 wt %, or 0.5 wt % to 2.0 wt %, or 1.0 wt % to 10 wt %, or 1.0 wt % to 5.0 wt %.

In various aspects, an oxygenate conversion reaction can be used to form a conversion effluent with an olefin content so that at least a portion of the conversion effluent can be used as at least a portion of the feed for oligomerization. In some aspects, the oxygenate conversion reaction can form a conversion effluent containing 5.0 wt % or more of C₂ olefins relative to the weight of the conversion effluent, or 10 wt % or more, or 15 wt % or more, or 20 wt % or more, such as up to 50 wt % or possibly still higher. In other aspects, the oxygenate conversion reaction can form a conversion effluent containing less than 15 wt % of C₂ olefins, or less than 10 wt %, or less than 5.0 wt %, such as down to having substantially no content of C₂ olefins (0.1 wt % or less). In addition to C₂ olefin content, the total olefin content, the C₂-C₈ olefin content and/or the C₃-C₈ olefin content of the conversion effluent can be characterized. In various aspects, the conversion effluent can have a total olefin content, C₂-C₈ olefin content, and/or C₃-C₈ olefin content of 15 wt % or more, or 25 wt % or more, or 40 wt % or more, or 50 wt % or more, or 60 wt % or more, or 80 wt % or more, such as up to the conversion effluent being substantially composed of olefins (up to 100 wt %).

### Examples—Combined Conversion of C₂ Olefins and C₃-C₈ Olefins with Reduced Production of C₃-C₆ Alkanes

A series of representative solid acid catalysts were tested for conversion of an olefinic feed. The catalyst performance testing experiments were run using a 0.334 inch (8.5 mm) internal diameter stainless steel reactor. For a typical experiment, 1.5 g of crushed and sieved solid acid catalyst (300-600 micron particle size) was loaded in a ~150 mm isothermal zone and diluted with 2-3 g SiCₓ for improved heat transfer. The dilution with inert particles assisted with maintaining temperature in the reactor in spite of the highly exothermic nature of the oligomerization reaction.

Typical reaction conditions were 900-1000 psig (60-70 barg), between 160° C. to 310° C. and a weight-hourly-space-velocity WHSV of 4 h⁻¹. The feeds used in the experiments contained 18 wt % to 20 wt % ethylene, 53 wt % to 55 wt % propylene, 15 wt % to 17 wt % 1-butene, 6 wt % to 7 wt % 1-pentene, 2 wt % to 3 wt % 1-hexene, 1 wt % to 2 wt % isobutylene and ~1 wt % of isobutane. Throughout the runs the temperature was adjusted to achieve various levels of C₃₊ olefin conversion. A variety of conversion levels were achieved, with a goal of having roughly 90% to 95% conversion of C₃₊ olefins. This avoided excessive alkane production while still providing high feed conversion. Feed and reactor product analysis were performed by high pressure into a dedicated online gas chromatograph equipped with two low thermal mass module columns for light olefin/paraffin (conversion calculation) and heavy hydrocarbon (C₂-C₁₆₊ carbon number distribution (CND)) analysis. To simplify CND analysis, products were fully hydrogenated online before isomer analysis using a Pt/Al₂O₃ catalyst. Corrected temperatures were determined according to Equation 1 above, for corrected temperature based on C₂-C₆ conversion.

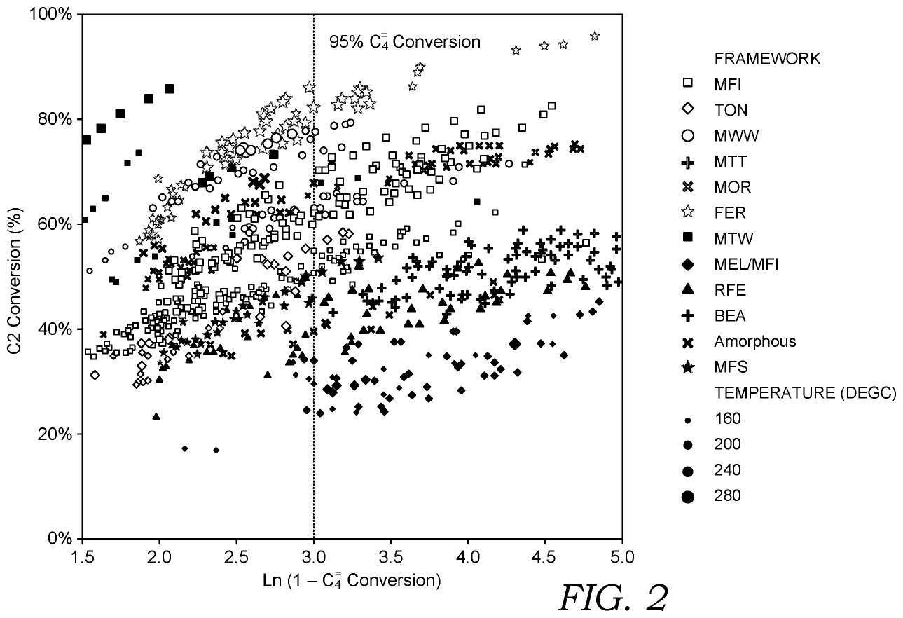

FIG. 2 shows C₂ olefin conversion versus the natural log of (1-C₄ olefin conversion). Using the natural log of “1-conversion” is a surrogate for the reaction rate. As shown in FIG. 2, the catalysts with MTW, MEL/MFI, MFS, MTT, and/or MFI framework all provide the ability to perform conversion of both C₂ and C₄ conversion.

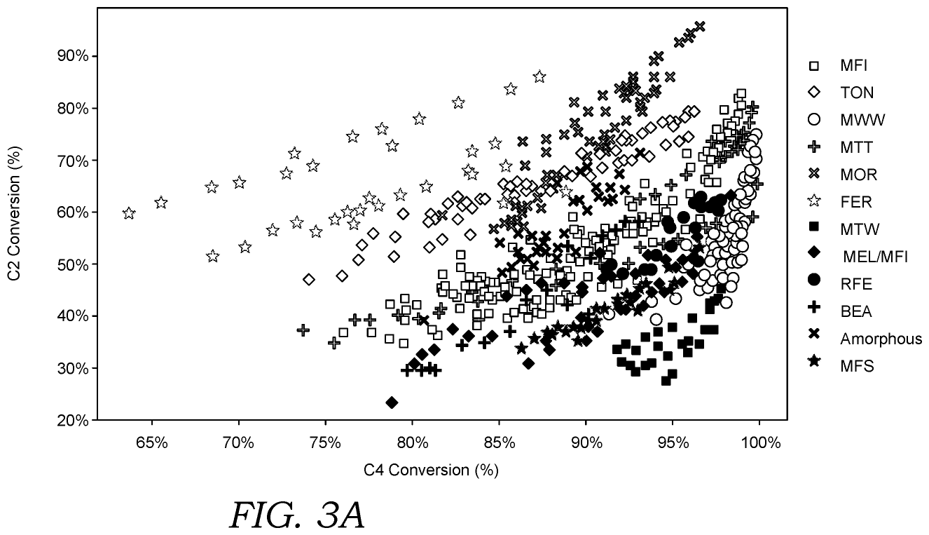

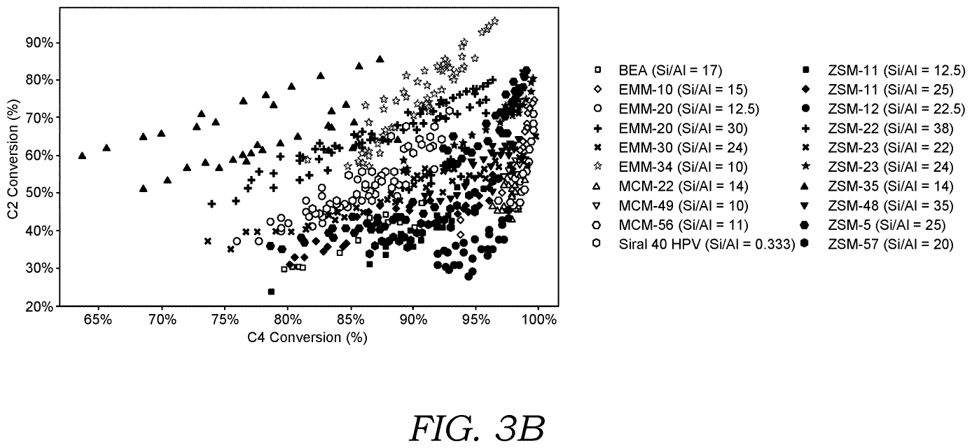

FIG. 3A and FIG. 3B show C₂ conversion and C₄ conversion for various types of zeolitic frameworks. In addition to catalysts based on MTW, MEL/MFI, MFS, MTT, and/or MFI, FIG. 2 and FIG. 3 also show MOR (mordenite), FER (ZSM-35), RFE (ZSM-48), *BEA (zeolite Beta), MWW (EMM-10), TON (ZSM-22) and an amorphous silica-alumina (Siral 40). Here, C₄ conversion is used as a proxy for conversion of C₃-C₈ olefins. It is understood that conventionally, C₃ olefins will be converted more readily than C₄ olefins during oligomerization. Additionally, as a practical matter, for most sources of olefins for oligomerization, the quantities of C₅₊ olefins will typically be relatively smaller than the content of C₂, C₃, or C₄ olefins. Thus, the ability to perform conversion of both C₂ and C₄ olefins corresponds to the ability to generally oligomerize olefinic feeds to fuel boiling range compounds with high conversion.

FIG. 3A shows C₂ conversion versus C₄ conversion for catalysts with various framework types. Generally, as shown in FIG. 3A, a catalyst having an MTW, MEL/MFI, MFS, MTT, and/or MFI framework provides meaningful levels of conversion for both C₂ olefins and C₄ olefins, but the amount of C₂ conversion relative to C₄ conversion varies. The C₂ conversion relative to C₄ conversion is relatively low for the catalysts having MEL/MFI, MFS, or MTW framework. By contrast, MTT and MFI framework catalysts provide relatively high C₂ conversion relative to C₄ conversion. It is noted that catalysts having MOR, FER, and TON frameworks also have relatively high conversion of both C₂ and C₄ olefins.

FIG. 3B shows example zeolites having the zeolite frameworks shown in FIG. 3A, along with the silica to alumina ratio for each zeolite. It is noted that EMM-34 corresponds to meso-mordenite (MOR), EMM-20 corresponds to an MFI framework material, and EMM-30 corresponds to a MEL/MFI framework material.

Additionally, FIG. 3A and FIG. 3B show C₂ conversion and C₄ conversion for various types of zeolitic frameworks. In addition to catalysts based on MOR, MTT, FER, TON, RFE (ZSM-48) and MFI, FIG. 3A and FIG. 3B also show MEL/MFI (ZSM-11), *BEA (zeolite Beta), MWW (EMM-10), and an amorphous silica-alumina (Siral 40). Here, C₄ conversion is used as a proxy for conversion of C₃-C₈ olefins. It is understood that conventionally, C₃ olefins will be converted more readily than C₄ olefins during oligomerization. Additionally, as a practical matter, for most sources of olefins for oligomerization, the quantities of C₅₊ olefins will typically be relatively smaller than the content of C₂, C₃, or C₄ olefins. Thus, the ability to perform high conversion of both C₂ and C₄ olefins corresponds to the ability to generally oligomerize olefinic feeds to fuel boiling range compounds with high conversion.

It has been discovered that catalysts based on framework types MOR, MTT, FER, TON, RFE, and MFI can provide unexpectedly high C₂ olefin conversion while also providing substantial C₄ conversion. This is beneficial for allowing single stage conversion of mixed streams of olefins that have substantial C₂ content. FIG. 3A and FIG. 3B show C₂ conversion versus C₄ conversion for catalysts with various framework types. As shown in FIG. 3A, catalysts having an MOR, MTT, FER, TON, RFE, and/or MFI framework type provide unexpectedly high C₂ conversion while still also providing substantial C₄ conversion.

FIG. 3B shows example zeolites along with the silica to alumina ratio for each zeolite. It is noted that EMM-34 corresponds to meso-mordenite (MOR), while EMM-20 and EMM-30 correspond to MFI framework materials. FIG. 3B shows that the zeolites having an MOR, MTT, FER, TON, RFE, and/or MFI framework structure provide unexpectedly high C₂ conversion while also providing substantial C₄ conversion.

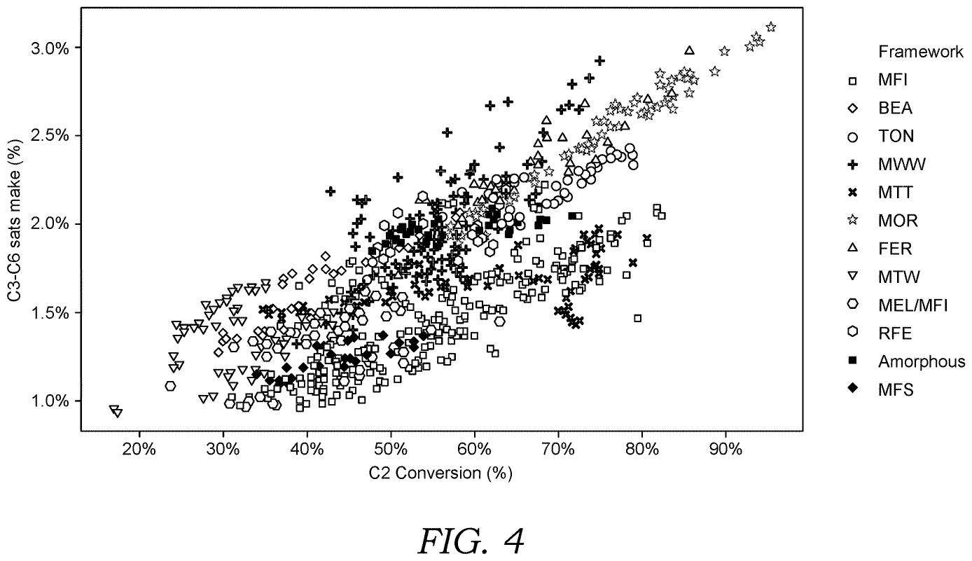

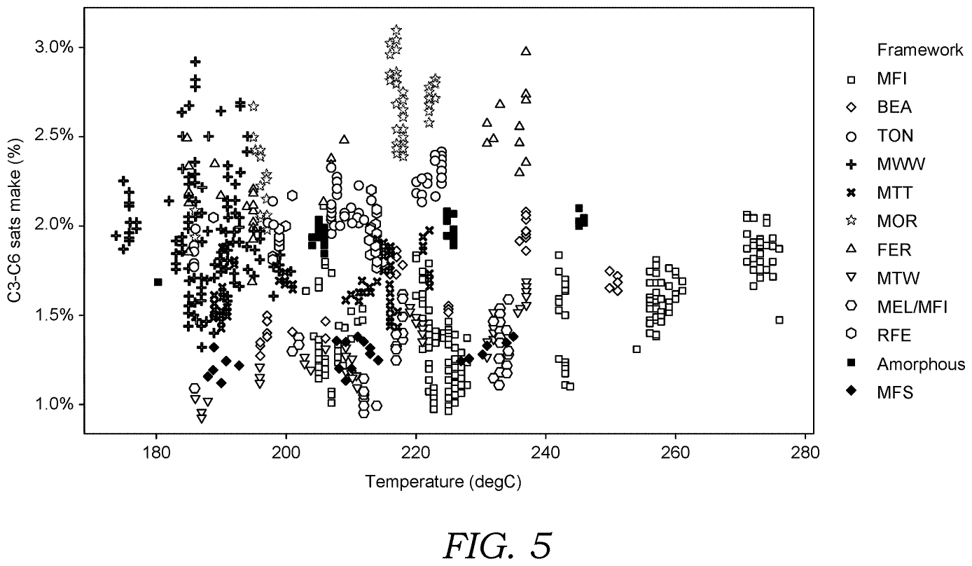

FIG. 4 and FIG. 5 illustrate that converting C₂ and C₃-C₈ olefins using catalysts having a MTW, MEL/MFI, MFS, MTT, and/or MFI framework results in unexpectedly low production of C₃-C₆ saturates (alkanes). As shown in FIG. 4 and FIG. 5, catalysts with a MTW, MEL/MFI, MFS, MTT, and/or MFI framework can have a selectivity for formation of C₃-C₆ alkanes of 2.0 wt % or less, or 1.5 wt % or less, or 1.0 wt % or less, such as down to 0.3 wt % or possibly still lower. The low selectivity for C₃-C₆ alkanes at high C₂ conversion is more pronounced for MTT and MFI framework catalysts. This low level of selectivity can be achieved while still providing conversion (single pass basis) of 20 wt % or more of C₂ olefins, or 35 wt % or more, or 50 wt % or more, or 60 wt % or more, such as up to 80 wt % or possibly still higher. Such C₃-C₆ paraffins are outside of the boiling range for the desired distillate fuel products and cannot undergo further oligomerization. It is desirable to reduce or minimize production of such paraffins in order to increase the net yield of distillate fuel products from the oligomerization reaction.

FIG. 5 shows additional details about the temperatures required for conversion of olefinic feeds using catalysts having various frameworks. The ability to operate at lower temperature provides a variety of advantages. For example, lower temperature operation typically enables longer run lengths. During an olefin oligomerization process, the catalyst typically deactivates over time, and the temperature of the reactor is increased to compensate for the loss of activity. However, it is usually not practical to increase the reaction temperature beyond about 310° C., as higher temperatures typically facilitate too many competing side reactions (including excess coke formation). Being able to start the run at a lower temperature allows for greater ability to increase temperature as a run progresses while avoiding processing at temperatures of greater than 310° C. Additionally, lower temperature operation tends to mitigate coke formation, one of the sources of deactivation.

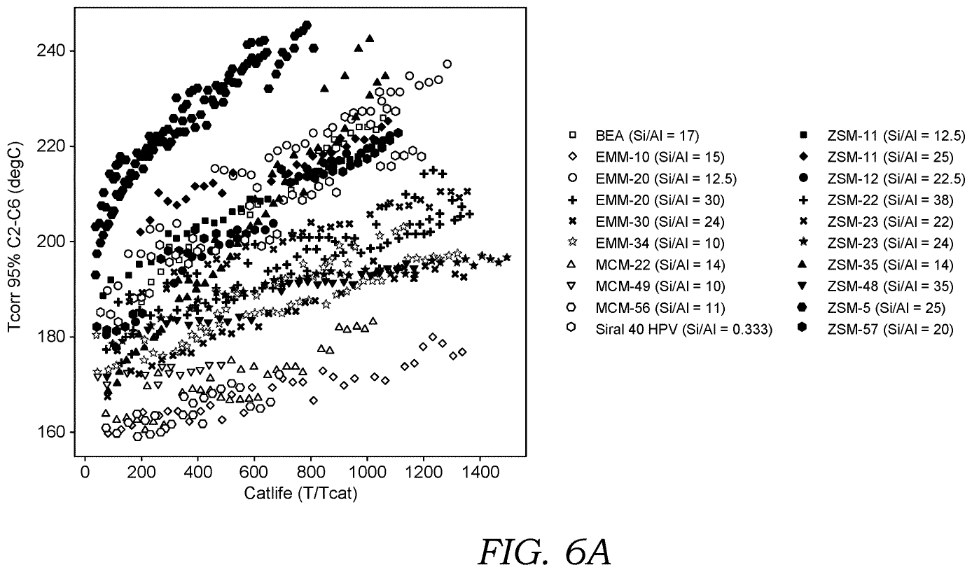

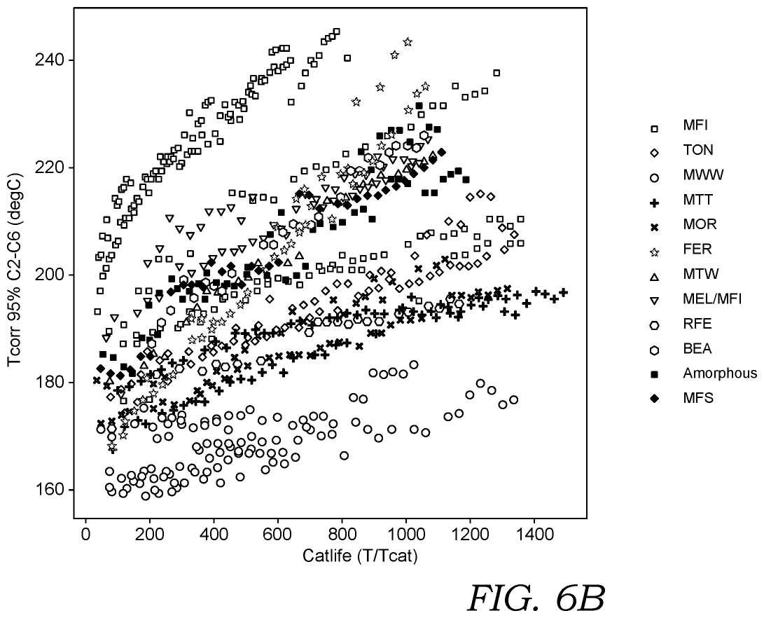

FIG. 6A and FIG. 6B shows the corrected temperature for achieving 95% conversion of C₂-C₆ olefins in the feed versus catalyst lifetime. The catalyst lifetime is defined as the mass of C₉-C₁₆₊ products generated by the catalyst during operation, with the run endpoint being defined as the time when the actual catalyst temperature during the run reaches 310° C. As shown in FIG. 6A and FIG. 6B, the MTW, MEL/MFI, MFS, MTT, and/or MFI catalysts can provide the conversion shown in FIG. 2, FIG. 3A, and FIG. 3B at reasonable conversion temperatures for providing a high catalyst lifetime.

Additionally, in addition to high C₂ olefin conversion while providing substantial C₄ olefin conversion, it has been discovered that catalysts having a MOR, MTT, and/or TON framework can provide the high C₂ olefin conversion at unexpectedly low temperatures. The ability to operate at lower temperature provides a variety of advantages. For example, lower temperature operation typically enables longer run lengths. During an olefin oligomerization process, the catalyst typically deactivates over time, and the temperature of the reactor is increased to compensate for the loss of activity. However, it is usually not practical to increase the reaction temperature beyond about 310° C., as higher temperatures typically facilitate too many competing side reactions (including excess coke formation) and, in case water/steam is used for reactor cooling, require more costly materials of construction to withstand the higher steam pressure. Being able to start the run at a lower temperature allows for greater ability to increase temperature as a run progresses while avoiding processing at temperatures of greater than 310° C. Additionally, lower temperature operation tends to mitigate coke formation, one of the sources of deactivation. As shown in FIG. 6A and FIG. 6B, the MWW framework catalyst provides the lowest corrected temperature. However, the MOR (EMM-34), MTT (ZSM-23), RFE (ZSM-48) and TON (ZSM-22) catalysts have only a modestly higher corrected temperature while also providing the superior C₂ conversion shown in FIG. 3A and FIG. 3B.

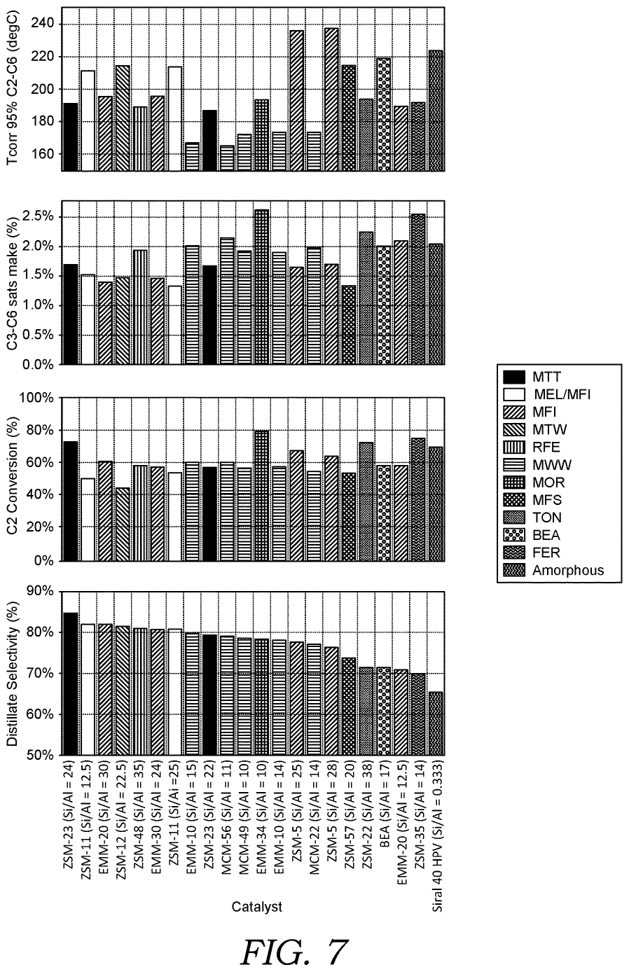

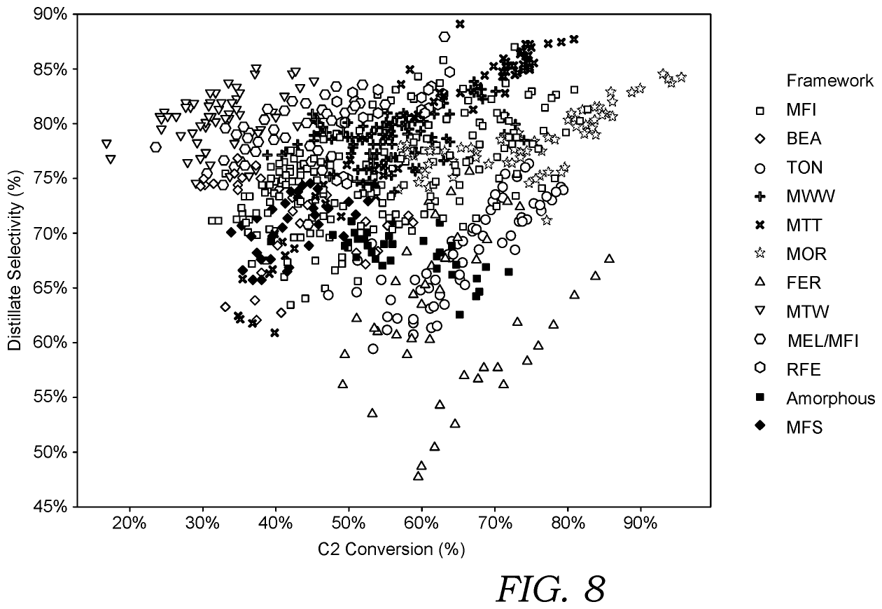

FIG. 7 and FIG. 8 show the selectivity for formation of distillate boiling range compounds relative to C₂ conversion. It is noted that in FIG. 7 and FIG. 8, MTT and MFI framework catalysts provide unexpectedly favorable combinations of C₂ conversion and distillate selectivity.

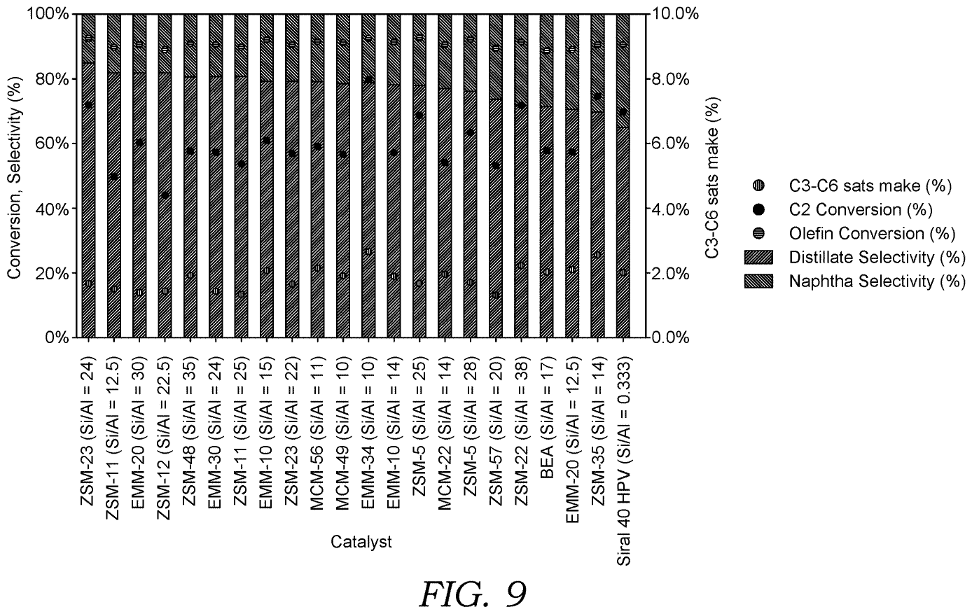

FIG. 9 provides a summary of the C₃-C₆ saturates (alkanes) make, C₂ conversion, naphtha (gasoline) selectivity, and distillate selectivity for oligomerization of the olefinic feed over various catalysts, averaged between 90 and 95% C₃₊ olefin conversion. In FIG. 9, the left vertical axis is from 0 to 100. This left vertical axis is used both for understanding the product selectivity values (bars) as well as the values for amount of C₂ conversion and total olefin conversion. The right vertical axis is for the C₃-C₆ selectivity. In FIG. 9, the data is organized based on decreasing selectivity for formation of distillate boiling range products (121° C. to 370° C.). Thus, the catalyst corresponding to ZSM-23 with a Si to Al ratio of 24 provided the highest distillate selectivity, while the catalyst corresponding to the amorphous silica alumina provided the lowest distillate selectivity. As shown in FIG. 9, catalysts having MTW (ZSM-12), MEL/MFI (ZSM-11, EMM-30), MTT (ZSM-23), MFS (ZSM-57), and/or MFI (ZSM-5, EMM-20) frameworks provided an unexpected combination of high distillate selectivity while also providing reduced or minimized formation of C₃-C₆ saturates. It is noted that for EMM-20, higher values of Si to Al ratio provided improved distillate selectivity.

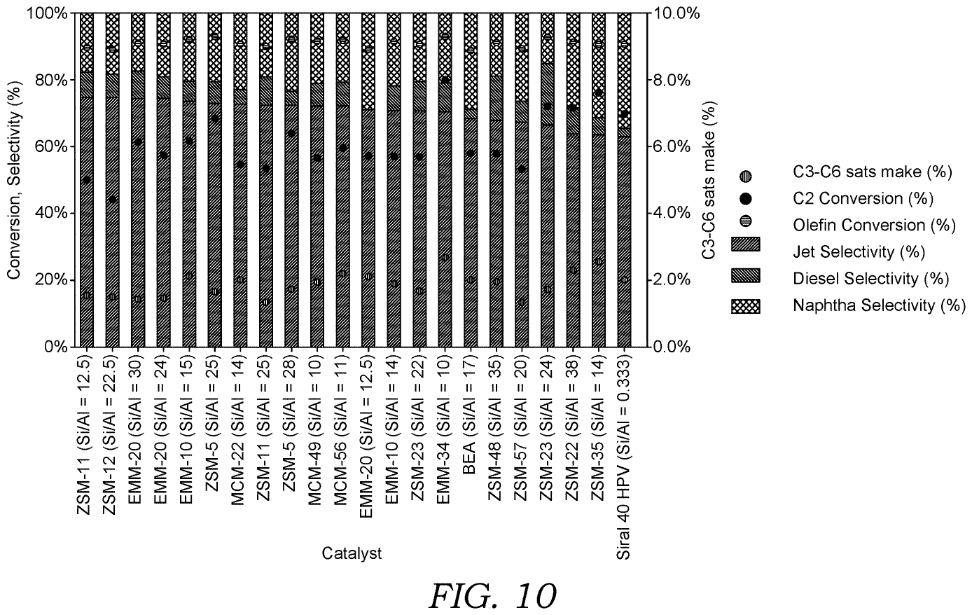

FIG. 10 provides a summary of selectivity for the same feed and catalysts shown in FIG. 9, but with the “distillate” product separately identified as a “jet” boiling range product and a “heavy diesel” boiling range product. In FIG. 10, the data is organized based on decreasing selectivity for formation of jet boiling range products (121° C. to 300° C.). As shown in FIG. 10, the catalysts that produced the highest selectivity for jet boiling range products (121° C. to 300° C.) products do not directly correspond to the catalysts that produced the highest selectivity for distillate boiling range products (121° C. to 370° C.). In particular, in FIG. 9, catalysts having MTT framework (ZSM-23) provided some of the highest distillate selectivities, but this is due in part to MTT framework catalysts having an unexpectedly higher selectivity for production of heavy diesel (300° C. to 370° C.), with a somewhat lower selectivity for production of jet boiling range products. Similarly, the MFI framework catalysts (ZSM-5, EMM-20) also have a higher selectivity for heavy diesel products. By contrast, catalysts having MTW (ZSM-12) and MEL/MFI (ZSM-11, EMM-30) frameworks had unexpectedly high jet selectivity, while having lower selectivity for heavy diesel.

### Examples—Conversion of C₃-C₈ Olefinic Feeds

In some aspects, MTW, MEL/MFI, MFS, MTT, and/or MFI catalysts can also be used for conversion of olefinic feeds that contain C₃-C₈ olefins, but that have a reduced or minimized content of C₂ olefins, such as 5.0 wt % or less. It is noted that for this group of experiments, formulated catalysts that include some amount of binder material were used, as opposed to the zeolite crystals that were used to generate the data shown in FIGS. 2-10. The amount of binder in these catalysts varied based on the activity of the type of zeolite, and was selected to represent a commercially relevant formulation for the resulting bound catalysts.

A series of representative solid acid catalysts were tested for conversion of an olefinic feed. The solid acid catalysts included ZSM-12 (MTW), ZSM-57 (MFS), ZSM-23 (MTT), ZSM-5 (MFI), MCM-49 (MWW), ZSM-22 (TON), and a commercially available amorphous silica-alumina.

Catalyst performance testing experiments were run using a 0.334 inch (8.5 mm) internal diameter stainless steel reactor and for a typical experiment 1.5-2 g of crushed and sieved solid acid catalyst formulated with binder (300-600 micron particle size) was loaded in a ~150 mm isothermal zone and diluted with 2-3 g SiCₓ for improved heat transfer. This allowed the reaction to be performed at a controlled temperature even though oligomerization is a highly exothermic reaction.

Typical reaction conditions were 900-1000 psig (60-70 barg), between 160-310° C. and a weight-hourly-space-velocity WHSV of 4 h⁻¹. Typical feed was 67% Propylene, 19% 1-butene, 7% 1-Pentene, 2% 1-Hexene, 1% Isobutylene and ~3-4% of isobutane. Throughout the runs the temperature was adjusted to achieve ~95% total olefin conversion. Feed and reactor product analysis were performed by high pressure into a dedicated online gas chromatograph equipped with two low thermal mass module columns for light olefin/paraffin (conversion calculation) and heavy hydrocarbon (C₂-C₁₆₊ carbon number distribution (CND)) analysis. To simplify CND analysis, products were fully hydrogenated online before isomer analysis using a Pt/Al₂O₃ catalyst. Jet selectivity was defined as C₉₊ Selectivity-C₁₆₊ Selectivity (the difference between C₉₊ selectivity and C₁₆₊ selectivity). Distillate selectivity corresponds to C₉₊ selectivity.

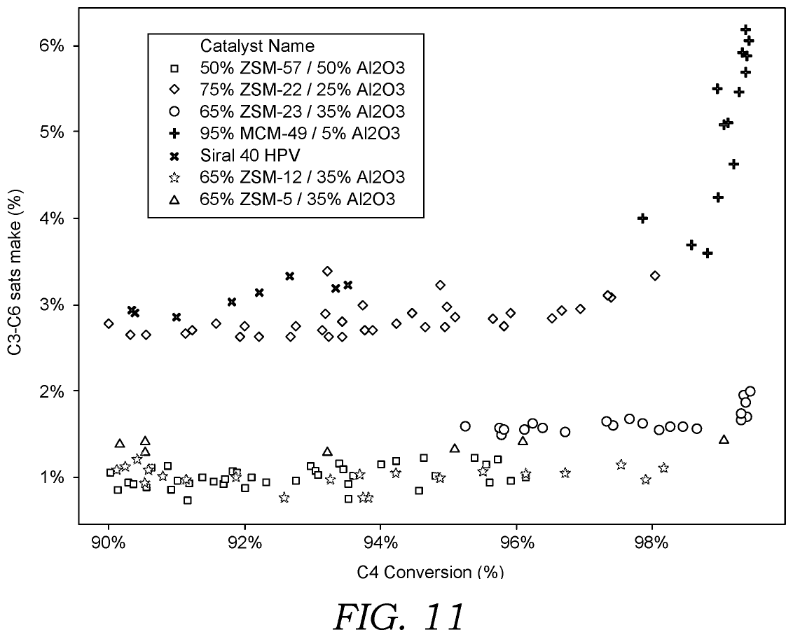

FIG. 11 shows C₃-C₆ saturates (alkanes) production relative to C₄ conversion. As shown in FIG. 11, at conversion levels between 90 wt % conversion of C₄ to 99 wt % conversion of C₄, the catalysts containing the MTW, MFS, MTT, and MFI frameworks provided substantially lower production of C₃-C₆ saturates. It is noted that although MEL frameworks are not shown in FIG. 11, in testing with zeolite crystals (not formulated), MEL zeolites also provided low C₃-C₆ saturates.

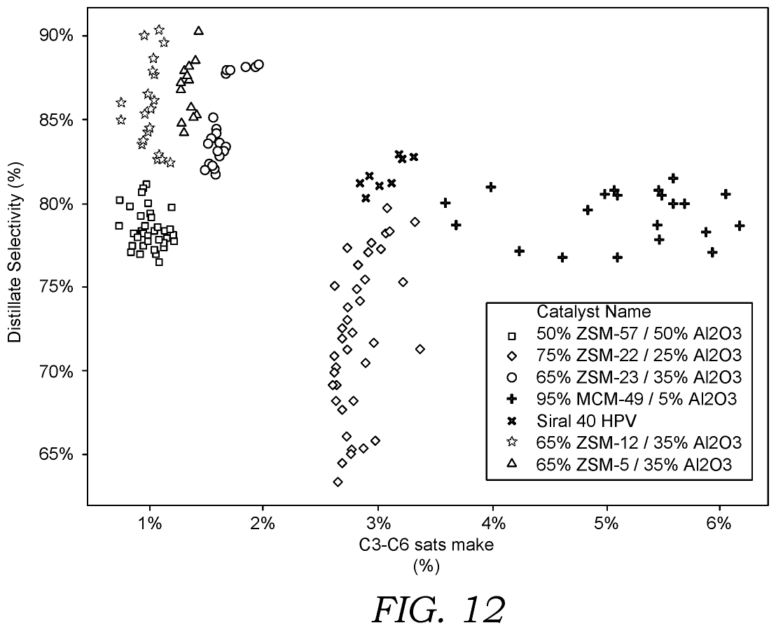

FIG. 12 shows distillate selectivity relative to C₃-C₆ saturates production for the various catalysts. As shown in FIG. 12, the distillate selectivity for the MTW, MFS, MTT, and MFI framework catalysts is comparable to or better than the distillate selectivity for the solid acid catalysts, while also providing substantially reduced production of C₃-C₆ alkanes. This combination of high distillate selectivity with low production of C₃-C₆ alkanes is unexpected.

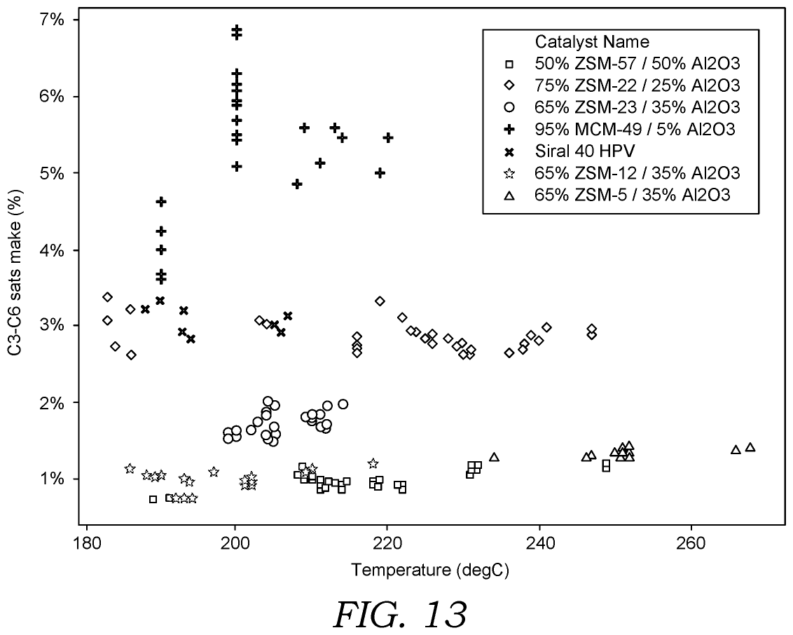

To further illustrate the unexpected nature of the low C₃-C₆ saturates production for MTW, MFS, MTT, and MFI framework catalysts, FIG. 13 shows C₃-C₆ saturates production relative to reactor temperature. As shown in FIG. 13, the low production of C₃-C₆ saturates for MTW, MFS, MTT, and MFI framework catalysts is maintained substantially independent of the reactor temperature.

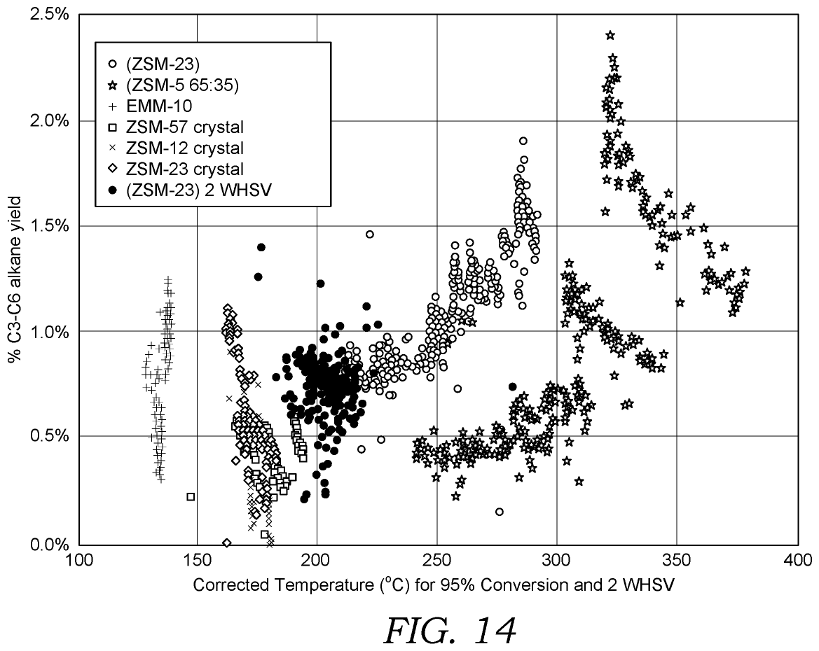

It is noted that an additional catalyst with unexpectedly low C₃-C₆ saturates production was also discovered. FIG. 14 shows additional results for C₃-C₆ saturates formation for several solid acid catalysts. For the data in FIG. 13, the solid acid catalysts were exposed to the olefinic feed under conditions sufficient to achieve at least 70 wt % conversion of the C₄ olefins in the olefinic feed. The olefinic feed was a mixture of olefins corresponding to 30 vol % propylene, 17.5 vol % butenes, 16.5 vol % pentenes, and 36 vol % octenes. Equation 2 above was then used to determine the corrected temperature based on C₄ conversion. The runs with each solid acid catalyst were sampled multiple times.

It was determined that butene was a good indicator of catalyst performance and the olefin conversion process has been shown to operate effectively at 95% butene conversion. With this composition of feed, when butene conversion was 95%, propylene conversion was typically 99+% conversion because it has less transport restrictions and therefore diffuses very quickly through the pores of the zeolite to the active catalytic sites. Pentene conversion was ~50-70 wt %, while octene conversion was less than ~30 wt %.

In FIG. 14, all of the catalysts tested provided a selectivity for C₃-C₆ saturates formation of less than 2.0 wt %. It is noted, however, that the EMM-10 catalyst allowed for unusually low reaction temperatures of 120° C. or less while still providing substantial conversion of the feed. This appeared to allow for reduced production of C₃-C₆ saturates relative to the MCM-49 catalyst shown in FIGS. 11-13.

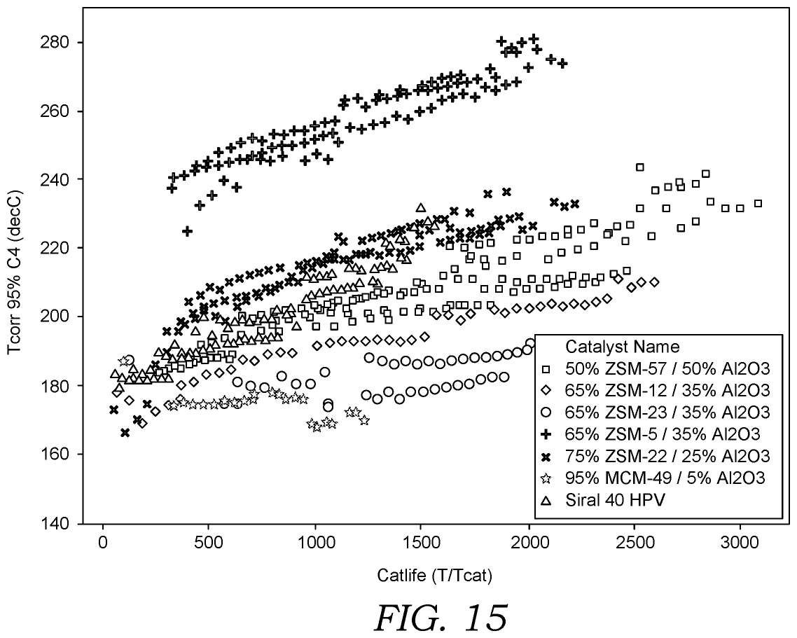

FIG. 15 shows the corrected temperature for 95 wt % conversion of C₃-C₆ olefins versus catalyst life for various catalysts using a model feed for oligomerization.

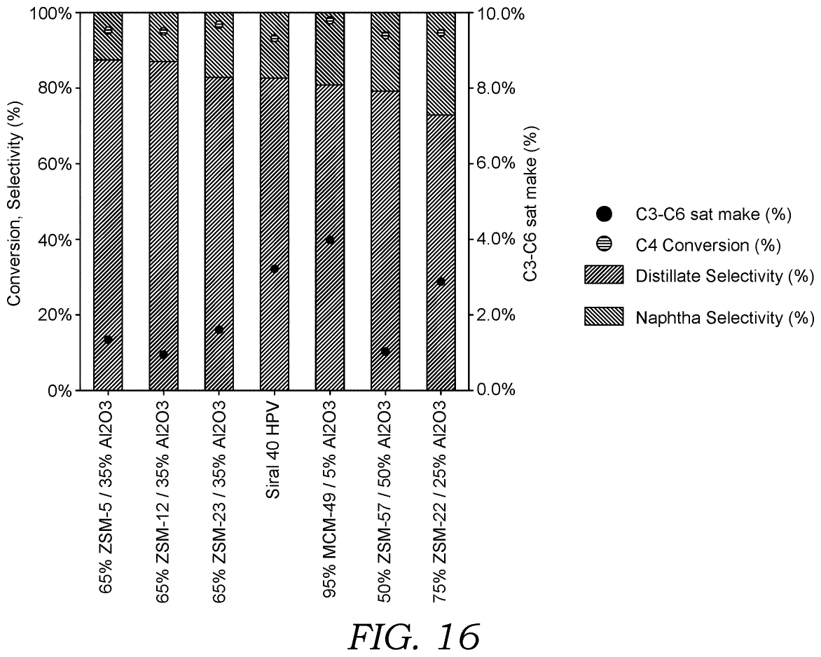

FIG. 16 shows the C₃-C₆ saturates (alkanes) make, C₄ conversion and distillate and naphtha selectivity for oligomerization of the C₃-C₆ olefinic feed over various catalysts. Data averaged between 90 and 95% olefin conversion. As shown in FIG. 16, the catalysts based on MTT (ZSM-23), MTW (ZSM-12), MFI (ZSM-5, EMM-20), and MFS (ZSM-57) zeolites provided unexpectedly low selectivity for formation of C₃-C₆ saturates.

### Additional Embodiments

Embodiment 1. A method of converting a feed containing C₂-C₈ olefins, comprising: exposing a feed comprising 50 wt % or more of C₂-C₈ olefins to a catalyst comprising a zeolitic framework structure having a 1-dimensional or 3-dimensional largest pore channel under conversion conditions to form a conversion product comprising 70 wt % or more of 121° C.+ components and 2.0 wt % or less of C₃-C₆ alkanes, relative to a weight of olefins in the feed, the conversion conditions comprising single pass conversion of 30 wt % or more of C₂ olefins and 80 wt % or more of C₄ olefins, wherein a maximum diameter of a sphere that can be included in the largest pore channel is 5.0 Angstroms to 6.5 Angstroms.

Embodiment 2. The method of Embodiment 1, wherein the zeolitic framework structure further comprises an elliptical 8-membered ring pore channel having a pore channel size of 2.2 Angstroms to 3.5 Angstroms for a minor elliptical axis and 4.0 Angstroms to 6.0 Angstroms for a major elliptical axis.

Embodiment 3. A method of converting a feed containing C₂-C₈ olefins, comprising: exposing a feed comprising 5.0 wt % or more of C₂ olefins and 25 wt % or more of C₂-C₈ olefins to a catalyst comprising a zeolitic framework structure under conversion conditions to form a conversion product comprising 70 wt % or more of 121° C.+ components, relative to a weight of olefins in the feed, the conversion conditions comprising single pass conversion of 50 wt % or more of C₂ olefins and 80 wt % or more of C₄ olefins, wherein the catalyst comprises i) a zeolitic framework structure comprising an elliptical 8-membered ring pore channel having a pore channel size of 2.2 Angstroms to 3.5 Angstroms for a minor elliptical axis and 4.0 Angstroms to 6.0 Angstroms for a major elliptical axis, the zeolitic framework structure being a 2-dimensional or 3-dimensional zeolitic framework structure; ii) a zeolitic framework structure where a maximum diameter of a sphere that can be included in a largest pore channel is 4.0 Angstroms to 6.0 Angstroms; or iii) a combination of i) and ii).

Embodiment 4. The method of Embodiment 3, wherein the catalyst comprises a zeolitic framework structure having a 1-dimensional largest pore channel, where the maximum diameter of a sphere that can be included in the largest pore channel is 4.0 Angstroms to 6.0 Angstroms.

Embodiment 5. The method of Embodiment 3 or 4, wherein the conversion product further comprises 2.0 wt % or less of C₃-C₆ alkanes.

Embodiment 6. The method of any of the above embodiments, wherein the zeolitic framework structure comprises a Si to Al ratio of 5 to 40, preferably 8 to 25.

Embodiment 7. The method of any of the above embodiments, wherein the catalyst comprises a particle size of 500 nm or less, or wherein the catalyst comprises a maximum diffusion length within the framework structure of 500 nm or less, or a combination thereof.

Embodiment 8. The method of any of the above embodiments, wherein the conversion conditions comprise a temperature of 120° C. to 300° C., a pressure of 0.1 MPa-a to 10 MPa-a, and a weight hourly space velocity of 0.05 hr⁻¹ to 5.0 hr⁻¹, the pressure optionally being 5.0 MPa-a to 7.5 MPa-a.

Embodiment 9. The method of any of the above embodiments, wherein the conversion conditions comprise conversion of 90 wt % or more of the C₄ olefins, or wherein the conversion conditions comprise conversion of 90 wt % or more of C₃-C₈ olefins, or a combination thereof.

Embodiment 10. The method of any of the above embodiments, wherein the conversion product comprises 1.5 wt % or less of C₃-C₆ alkanes.

Embodiment 11. The method of any of the above embodiments, wherein the conversion product comprises 70 wt % or more of C₉-C₁₆₊ components, relative to a weight of olefins in the feed.

Embodiment 12. The method of any of the above embodiments, wherein the conversion conditions comprise a corrected temperature of 150° C. to 240° C. for 95 wt % conversion of C₄ olefins at a WHSV of 2.0 hr⁻¹; or wherein the conversion conditions comprise a corrected temperature of 150° C. to 240° C. for 95 wt % conversion of C₂-C₆ olefins at a WHSV of 2.0 hr⁻¹; or a combination thereof.

Embodiment 13. The method of any of the above embodiments, wherein the method further comprises: separating the conversion product to form one or more light fractions comprising C₃-C₈ components and one or more distillate product fractions comprising C₉₊ components, wherein the feed comprises at least a portion of the one or more light fractions.

Embodiment 14. The method of any of the above embodiments, wherein the feed has a molar ratio of olefins to isoparaffins of 1.0 or higher; or wherein the feed comprises no isoparaffins; or wherein the feed comprises no aromatics; or a combination of two or more thereof.

Embodiment 15. The method of any of the above embodiments, a) wherein the catalyst comprises 0.75 wt % or less of Group 5-10 and Group 13 non-noble metals supported on the catalyst, or 0.5 wt % or less, b) wherein the catalyst comprises 0.05 wt % or less of Pt, Pd, or a combination thereof supported on the catalyst, or c) a combination of a) and b).

Embodiment 16. The method of any Embodiments 1 to 14, wherein the catalyst comprises 0.5 wt % or less of Group 5-14 non-noble metals supported on the catalyst, or wherein the catalyst comprises 0.01 wt % or less of Group 8-11 noble metals supported on the catalyst, or a combination thereof.

Embodiment 17. The method of any of the above embodiments, further comprising exposing an oxygenate feedstock comprising one or more alcohols to a conversion catalyst under conversion conditions to form a conversion effluent, the feed comprising at least a portion of the conversion effluent.

Embodiment 18. The method of Embodiment 17, wherein the conversion effluent comprises 5.0 wt % or more (or 10 wt % or more) of C₂ olefins and 50 wt % or more of C₂-C₈ olefins; or wherein the conversion effluent comprises less than 10 wt % of C₂ olefins (or less than 5.0 wt %) and 50 wt % or more of C₂-C₈ olefins.

Certain features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values take into account experimental error and variations that would be expected by a person having ordinary skill in the art.

The foregoing description of the disclosure illustrates and describes the present methodologies. Additionally, the disclosure shows and describes exemplary methods, but it is to be understood that various other combinations, modifications, and environments may be employed and the present methods are capable of changes or modifications within the scope of the concept as expressed herein, commensurate with the above teachings and/or the skill or knowledge of the relevant art.

We claim:

1. A method of converting a feed containing C₂-C₈ olefins, comprising:
exposing a feed comprising 50 wt % or more of C₂-C₈ olefins to a catalyst comprising a zeolitic framework structure having a 1-dimensional or 3-dimensional largest pore channel under conversion conditions to form a conversion product comprising 70 wt % or more of 121° C.+ components and 2.0 wt % or less of C₃-C₆ alkanes, relative to a weight of olefins in the feed, the conversion conditions comprising single pass conversion of 30 wt % or more of C₂ olefins and 80 wt % or more of C₄ olefins,
wherein a maximum diameter of a sphere that can be included in the largest pore channel is 5.0 Angstroms to 6.5 Angstroms.

2. The method of claim 1, wherein the zeolitic framework structure further comprises an elliptical 8-membered ring pore channel having a pore channel size of 2.2 Angstroms to 3.5 Angstroms for a minor elliptical axis and 4.0 Angstroms to 6.0 Angstroms for a major elliptical axis.

3. The method of claim 1, a) wherein the zeolitic framework structure comprises a Si to Al ratio of 5 to 40; b) wherein the catalyst comprises a particle size of 500 nm or less; c) wherein the catalyst comprises a maximum diffusion length within the framework structure of 500 nm or less; or d) a combination of two or more of a), b) and c).

4. The method of claim 1, wherein the conversion conditions comprise a temperature of 120° C. to 300° C., a pressure of 0.1 MPa-a to 10 MPa-a, and a weight hourly space velocity of 0.05 hr⁻¹ to 5.0 hr⁻¹.

5. The method of claim 1, wherein the conversion conditions comprise conversion of 90 wt % or more of C₃-C₈ olefins.

6. The method of claim 1, wherein the conversion product comprises 1.5 wt % or less of C₃-C₆ alkanes.

7. The method of claim 1, wherein the catalyst comprises 0.75 wt % or less of Group 5-10 and Group 13 non-noble metals supported on the catalyst, or 0.05 wt % or less of Pt, Pd, or C) a combination thereof, supported on the catalyst.

8. The method of claim 1, wherein the conversion product comprises 70 wt % or more of C₉-C₁₆₊ components, relative to a weight of olefins in the feed.

9. The method of claim 1, wherein the conversion conditions comprise a corrected temperature of 150° C. to 240° C. for 95 wt % conversion of C₂-C₆ olefins at a WHSV of 2.0 hr⁻¹.

10. The method of claim 1, wherein the method further comprises:
separating the conversion product to form one or more light fractions comprising C₃-C₈ components and one or more distillate product fractions comprising C₉₊ components, wherein the feed comprises at least a portion of the one or more light fractions.

11. The method of claim 1, wherein the feed has a molar ratio of olefins to isoparaffins of 1.0 or higher.

12. The method of claim 1, wherein the feed comprises no isoparaffins, or wherein the feed comprises no aromatics, or a combination thereof.

13. The method of claim 1, further comprising exposing an oxygenate feedstock comprising one or more alcohols to a conversion catalyst under conversion conditions to form a conversion effluent, the feed comprising at least a portion of the conversion effluent.

14. The method of claim 13, wherein the conversion effluent comprises 5.0 wt % or more (or 10 wt % or more) of C₂ olefins and 50 wt % or more of C₂-C₈ olefins; or wherein the conversion effluent comprises less than 10 wt % of C₂ olefins (or less than 5.0 wt %) and 50 wt % or more of C₂-C₈ olefins.

15. The method of claim 1, wherein the catalyst comprises an aluminosilicate.

16. A method of converting a feed containing C₂-C₈ olefins, comprising:
exposing a feed comprising 5.0 wt % or more of C₂ olefins and 25 wt % or more of C₂-C₈ olefins to a catalyst comprising a zeolitic framework structure under conversion conditions to form a conversion product comprising 70 wt % or more of 121° C.+ components, relative to a weight of olefins in the feed, the conversion conditions comprising single pass conversion of 50 wt % or more of C₂ olefins and 80 wt % or more of C₄ olefins,
wherein the catalyst comprises
i) a zeolitic framework structure comprising an elliptical 8-membered ring pore channel having a pore channel size of 2.2 Angstroms to 3.5 Angstroms for a minor elliptical axis and 4.0 Angstroms to 6.0 Angstroms for a major elliptical axis, the zeolitic framework structure being a 2-dimensional or 3-dimensional zeolitic framework structure;
ii) a zeolitic framework structure where a maximum diameter of a sphere that can be included in a largest pore channel is 4.0 Angstroms to 6.0 Angstroms; or
iii) a combination of i) and ii).

17. The method of claim 16, wherein the catalyst comprises a zeolitic framework structure having a 1-dimensional largest pore channel, where the maximum diameter of a sphere that can be included in the largest pore channel is 4.0 Angstroms to 6.0 Angstroms.

18. The method of claim 16, wherein the conversion product further comprises 2.0 wt % or less of C₃-C₆ alkanes.

19. The method of claim 16, wherein the conversion conditions comprise conversion of 90 wt % or more of the C₄ olefins.

20. The method of claim 16, a) wherein the zeolitic framework structure comprises a Si to Al ratio of 5 to 40; b) wherein the catalyst comprises a particle size of 500 nm or less; c) wherein the catalyst comprises a maximum diffusion length within the framework structure of 500 nm or less; or d) a combination of two or more of a), b) and c).

21. The method of claim 16, further comprising exposing an oxygenate feedstock comprising one or more alcohols to a conversion catalyst under conversion conditions to form a conversion effluent, the feed comprising at least a portion of the conversion effluent.

22. The method of claim 16, wherein the conversion conditions comprise a corrected temperature of 150° C. to 240° C. for 95 wt % conversion of C₄ olefins at a WHSV of 2.0 hr⁻¹.

23. The method of claim 16, i) wherein the catalyst comprises 0.5 wt % or less of Group 5-10 and Group 13 non-noble metals supported on the catalyst; ii) wherein the catalyst comprises 0.5 wt % or less of Group 5-14 non-noble metals supported on the catalyst; iii) wherein the catalyst comprises 0.01 wt % or less of Group 8-11 noble metals supported on the catalyst; or iv) a combination of two or more of i), ii), and iii).

24. The method of claim 16, wherein the conversion product comprises 70 wt % or more of C₉-C₁₆₊ components, relative to a weight of olefins in the feed.
